(19) United States
(12) Patent Application Publication
(10) Pub. No.: US 2026/0263820 A1
(43) Pub. Date: Sep. 10, 2026

(54) IMPLANTABLE NEUROSTIMULATOR HAVING POWER CONTROL AND THERMAL REGULATION AND METHODS OF USE

(71) Applicant: SetPoint Medical Corporation, Valencia, CA (US)

(72) Inventors: Michael A. FALTYS, Valencia, CA (US); Jesse M. SIMON, Los Angeles, CA (US)

(21) Appl. No.: 19/373,589

(22) Filed: Oct. 29, 2025

Related U.S. Application Data
(63) Continuation of application No. 17/751,505, filed on May 23, 2022, now Pat. No. 12,472,368.
Application No. 17/751,505 is a division of application No. 16/728,880, filed on Dec. 27, 2019, now Pat. No. 11,383,091.
Application No. 16/728,880 is a division of application No. 15/415,764, filed on Jan. 25, 2017, now Pat. No. 10,583,304.
(60) Provisional application No. 62/286,951, filed on Jan. 25, 2016.
(60) Provisional application No. 62/286,955, filed on Jan. 25, 2016.
(60) Provisional application No. 62/340,937, filed on May 24, 2016.

(51) Int. Cl. A61N 1/378; A61N 1/36; A61N 1/372; A61N 1/375

(52) U.S. Cl. CPC A61N 1/3787; A61N 1/3606; A61N 1/37205; A61N 1/3756; A61N 1/36053

(57) ABSTRACT

Leadless, implantable microstimulators for treating chronic inflammation. These devices can include a static magnetic field detector (e.g., non-Hall effect sensors/detectors, including those based on a Wiegand effect or generating pulses at a predetermined frequency range and using a detection circuit to determine the decay rate of the pulses), to trigger an emergency shut off of the microstimulator. Also described are methods and apparatuses for regulating the temperature of an implant based applied power from a charger (e.g., voltage across the charger when unloaded and when loaded by the implant) to yield a power control loop correlated with the power drawn by the implant to determine temperature of the implant. A negotiation protocol can exchange data between the charger and the implant (e.g., type of charger, type of implant, nature of the coupling between the two, etc.) to set target power control loop parameters to estimate and regulate implant temperature.

### CROSS REFERENCE TO RELATED APPLICATIONS

This patent application is a continuation of U.S. patent application Ser. No. 17/751,505, titled “IMPLANTABLE NEUROSTIMULATOR HAVING POWER CONTROL AND THERMAL REGULATION AND METHODS OF USE,” filed on May 23, 2022, now U.S. Patent Application Publication No. 2022/0280797, which is a division of U.S. patent application Ser. No. 16/728,880, titled “IMPLANTABLE NEUROSTIMULATOR HAVING POWER CONTROL AND THERMAL REGULATION AND METHODS OF USE,” filed on Dec. 27, 2019, now U.S. Pat. No. 11,383,091, which is a division of U.S. patent application Ser. No. 15/415,764, titled “IMPLANTABLE NEUROSTIMULATOR HAVING POWER CONTROL AND THERMAL REGULATION AND METHODS OF USE,” filed on Jan. 25, 2017, now U.S. Pat. No. 10,583,304, which claims priority to U.S. Provisional Patent Application No. 62/286,951, titled “POWER CONTROL AND THERMAL REGULATION OF AN IMPLANTABLE NEURO STIMULATOR,” filed on Jan. 25, 2016; U.S. Provisional Patent Application No. 62/340,937, titled “POWER CONTROL AND THERMAL REGULATION OF AN IMPLANTABLE NEURO STIMULATOR,” filed on May 24, 2016; and U.S. Provisional Patent Application No. 62/286,955, titled “IMPLANTABLE MICROSTIMULATORS,” filed on Jan. 25, 2016, each of which is herein incorporated by reference in its entirety. This patent application may be related to U.S. patent application Ser. No. 14/887,192, titled “NEURAL STIMULATION DEVICES AND SYSTEMS FOR TREATMENT OF CHRONIC INFLAMMATION,” filed Oct. 19, 2015, which is a continuation of U.S. patent application Ser. No. 14/508,940, titled “NEURAL STIMULATION DEVICES AND SYSTEMS FOR TREATMENT OF CHRONIC INFLAMMATION,” filed Oct. 7, 2014, now U.S. Pat. No. 9,162,064, which is a continuation of U.S. patent application Ser. No. 14/082,047, titled “NEURAL STIMULATION DEVICES AND SYSTEMS FOR TREATMENT OF CHRONIC INFLAMMATION,” filed Nov. 15, 2013, now U.S. Pat. No. 8,855,767, which is a divisional of U.S. patent application Ser. No. 12/978,250, titled “NEURAL STIMULATION DEVICES AND SYSTEMS FOR TREATMENT OF CHRONIC INFLAMMATION,” filed on Dec. 23, 2010, now U.S. Pat. No. 8,612,002, which claims priority to U.S. Provisional Patent Application Nos. 61/289,946, titled “LEADLESS CUFF MICROSTIMULATOR STIMULATOR,” filed on Dec. 23, 2009; and 61/306,849, titled “NEURAL STIMULATION DEVICES AND SYSTEMS FOR TREATMENT OF CHRONIC INFLAMMATION,” filed on Feb. 22, 2010. Also incorporated by reference herein is: abandoned U.S. patent application Ser. No. 12/874,171, titled “PRESCRIPTION PAD FOR TREATMENT OF INFLAMMATORY DISORDERS” filed Sep. 1, 2010, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are implantable microstimulators (e.g., neural stimulation systems and devices) that may include a static magnetic field detector for emergency shut-off of the implant. These implants may be configured to negotiate with an external charger (via inductive coils) to control charging power based at least in part an estimate of implant temperature, e.g., surface temperature, derived by the charger. The implant may alternatively or additionally negotiate the charging level provided by the charger to extend communication between the implant and the charger. In particular, the apparatuses (systems and devices, including implants and/or chargers) described herein may be configured for electrically stimulating one or more nerves (e.g., the vagus nerve) to treat chronic inflammation by modulation of the inflammatory response (via the nicotinic cholinergic anti-inflammatory pathway).

### BACKGROUND

Implantable electrical stimulation devices have been developed for therapeutic treatment of a wide variety of diseases and disorders. For example, implantable cardioverter defibrillators (ICDs) have been used in the treatment of various cardiac conditions. Spinal cord stimulators (SCS), or dorsal column stimulators (DCS), have been used in the treatment of chronic pain disorders including failed back syndrome, complex regional pain syndrome, and peripheral neuropathy. Peripheral nerve stimulation (PNS) systems have been used in the treatment of chronic pain syndromes and other diseases and disorders. Functional electrical stimulation (FES) systems have been used to restore some functionality to otherwise paralyzed extremities in spinal cord injury patients.

Typical implantable electrical stimulation systems may include one or more programmable electrodes on a lead that are connected to an implantable pulse generator (IPG) that contains a power source and stimulation circuitry. However, these systems can be difficult and/or time consuming to implant, as the electrodes and the IPG are usually implanted in separate areas and therefore the lead must be tunneled through body tissue to connect the IPG to the electrodes. Also, leads are susceptible to mechanical damage over time, particularly as they are usually thin and long.

Recently, small implantable neural stimulator technology, i.e. microstimulators, having integral electrodes attached to the body of a stimulator has been developed to address the disadvantages described above. This technology allows the typical IPG, lead and electrodes described above to be replaced with a single integral device. Integration of the lead has several advantages including reduction of surgery time by eliminating, for example, the need for implanting the electrodes and IPG in separate places, the need for a device pocket, the need for tunneling to the electrode site, and requirements for strain relief ties on the lead itself. Reliability may therefore be increased significantly, especially in soft tissue and across joints because active components, such as lead wires, are now part of the rigid structure and are not subject to the mechanical damage due to repeated bending or flexing over time.

Unfortunately, the currently developed leadless devices tend to be larger and more massive than desirable, and even than traditional electrode/lead assemblies, making it difficult to stably position such devices in the proper position with respect to the nerve. Without device stability, the nerve and/or surrounding muscle or tissue can be damaged due to movement of the assembly. Further these devices require long charging times, and are often difficult to control (e.g., program) and regulate. There remains a need for leadless integral microstimulator devices that can be stably positioned on the nerve and regulate their power, including power delivered by an outside charger to inductively charge the implant.

The power requirement of implantable neurostimulators may be highly limiting. The greater the power required, the longer the charging time, and the larger the implant (e.g., battery, capacitor, etc.) must be. In addition, because these devices are implanted, they must be protected or constrained from heating above internal body temperature more than a minimum amount (e.g., 2° C.), or risk damaging tissues. This may be particularly challenging when inductive charging is used to charge the implant.

Moreover, there might be unexpected situations such as circuit dysfunction or failure, sudden health condition change of the patient, unusual environment, thermistor failure, etc. A manual shut-off of the microstimulator may be needed in such emergency situations. There is a need for a manual shut off of the implantable microstimulator to protect the patients in case of emergency.

Described herein are microstimulators (MS, also referred to herein as neurostimulators, microregulators, MRs, etc.) and methods of using them that may address some of the needs identified above.

### SUMMARY OF THE DISCLOSURE

Described herein are systems for the treatment of chronic inflammatory disorders that include an implantable microstimulator with a static magnetic field detector.

Any of these apparatuses may include a detector for detecting a static magnetic field of a predetermined strength to disable (e.g., turn “off”, suspend, disable or prevent stimulation from the device). For example, disclosed herein include a leadless, implantable microstimulator devices (neurostimulators) for treating chronic inflammation that are configured to have an emergency shut-off that may be triggered by a predetermined static magnetic field that is externally applied near the implant. In particular, described herein are implants that are based on non-hall effect sensors for detecting the static magnetic field. This is because Hall effect sensors are typically larger and may not be appropriate for the very small, compact and low-energy neurostimulator implants described herein.

In general, the device can comprise a hermetically sealed capsule body, at least two electrically conductive capsule regions, wherein each region electrically connects to an electrode for applying stimulation to a vagus nerve. The implantable microstimulator device can comprise a resonator within the sealed capsule body. The resonator can comprise a coil and a capacitor configured to resonate at a predetermined frequency range.

The implantable microstimulator device can comprise a static magnetic field detector. The static magnetic field detector can comprise a low power pulse generator to generate pulses at the predetermined frequency range, the pulses configured to be introduced into the resonator, and a detection circuit configured to monitor a decay rate of the pulses. The implantable microstimulator device can further comprise a battery within the sealed capsule body, and an electronic assembly within the sealed capsule body. The electronic assembly comprises power management circuitry configured to receive power from the resonator to charge the battery, a microcontroller configured to control stimulation of the vagus nerve from the conductive capsule regions, and an emergency shut-off control configured to shut down the device when the decay rate increases to a predetermined threshold decay rate.

In some variations, a magnet can be placed near the implantable microstimulator to trigger the static magnetic field detector and cause an emergency shut off of the microstimulator. In some variations, the static magnetic field detector can comprise a resonator built in the microstimulator. The resonator can utilize an antenna, which is configured to receive power from an external charger. The antenna can comprise a coil of wire with a ferrite core to form an inductor with a defined inductance. This inductor can be coupled with a capacitor and a resistance to form a resonant circuit (RLC circuit). An external high quality NPO capacitor can be used to set the tank frequency. The frequency can be set to that of the radiated electric field to receive power and data from the external source. For example, the resonator can be a coil and capacitor configured to resonate at about 131 KHz+/−2%. The coil can be constructed with many turns of magnet wire with a target inductance of about 20 uH.

The static magnetic field detector can comprise a low power pulse generator. For example, the pulse generator can be an ultra-low power pulse generator based on a ring oscillator with the power between 1 nanowatt and 10 microwatts. In some other variations, the power of the pulse generator can be between 1 nanowatt and 1 microwatt. The low power pulse from the pulse generator can be introduced into the coil or the resonator RLC circuit. When the coil is tuned, the low power pulses can take a relatively long time to decay. But when a magnetic field is introduced to the coil, the coil is detuned. Therefore, the decay rate is much quicker (or time constant is smaller).

The static magnetic field detector can further comprise a detection circuit. The detection circuit can be configured to monitor the decay rate of the pulses or time constant of the resonator. The static magnetic field detector can be configured to shut down the microstimulator when the decay rate is increased to a predetermined value. The static magnetic field detector can be configured to shut down the microstimulator when the decay rate is 1.5, 2, 2.5, 3, 4, 5, 10 times of the previous value or any values therebetween. Values outside the above range are also possible. In some variation, the static magnetic field detector can be configured to shut down the microstimulator by using h a shorting switch. In some variations, the low power pulses can be introduced periodically, such as every second, to detect the presence of the magnetic field. In some other variations, the low pulses can be introduced continuously to monitoring the presence of the magnetic field.

In some variations the static magnetic field is detected using a Wiegand material (e.g., a material, such as a Wiegand wire exhibiting the Wiegand effect).

Methods of shutting down an implantable microstimulator device are also described herein. For example, described herein are methods of shut down an implantable microstimulator device by using a static magnetic field detector.

In some variations these methods may include the steps of: generating pulses by a low power pulse generator, introducing the pulses into an RLC resonator, monitoring a decay rate of the pulses, and shutting down the device when the decay rate increases to a predetermined threshold decay rate.

In some variations, the method of shutting down the device by the static magnetic field detector can comprise utilizing a built-in receiving coil of the microstimulator. In some other variations, the method can comprise utilizing another RLC resonator of the microstimulator.

In some variation, the method can comprise placing a magnet near the device. In some variation, the method can further comprise shutting down the device when the predetermined threshold decay rate increases 1.5, 2, 5, 10 times any values therebetween. In some variation, the method can comprise using a shorting switch configured to shut down the device.

In some variation, a power of the pulses from the low pulse generator is between 1 nanowatt and 10 microwatts. In some other variation, a power of the pulses is between 1 nanowatt and 1 microwatts. In some variation, the low power pulses are introduced periodically. In some other variation, the low power pulses are introduced continuously.

The above summary of the invention is not intended to describe each illustrated embodiment or every implementation of the present invention, but to highlight certain key features. The figures and the detailed description that follow more particularly exemplify these embodiments and features.

In addition or alternatively, any of the neurostimulators and chargers for charging these neurostimulators described herein include control circuitry that negotiates and controls the power applied by the charger to prevent overheating of the implanted neurostimulator, while still rapidly and efficiently charging the implant. For example, described herein are chargers for inductively charging a neurostimulator implanted within a portion of the patient's body. A charger may include: an energizer coil; an amplifier configured to drive an electrical current through the energizer coil to generate an electromagnetic field; and a controller comprising a power control loop configured to modulate the electrical current driven through the energizer coil based on an estimate of a tank voltage of the neurostimulator (PWRIN), to prevent a surface temperature of the neurostimulator from exceeding a predetermined threshold relative to the body temperature of the patient.

A charger for inductively charging a neurostimulator implanted within a portion of the patient's body may also or alternatively include: an energizer coil; an amplifier configured to drive an electrical current through the energizer coil to generate an electromagnetic field; and a controller comprising a power control loop configured to modulate the electrical current driven through the energizer coil such that a surface temperature of the neurostimulator remains within about 2 degrees Celsius of the body temperature of the patient.

The power control loop may be configured to modulate the electrical current driven through the energizer coil based on a measurement of a change in voltage across the energizer coil when the energizer coil is in an unloaded state and a loaded state from charging the neurostimulator.

The power control loop may be configured to modulate the electrical current driven through the energizer coil based on a power control loop parameter, wherein the power control loop parameter is based on a set power level of the amplifier and a measurement of a change in voltage across the energizer coil when the energizer coil is in an unloaded state and a loaded state from charging the neurostimulator.

In some variations, the set power level and change in voltage may be multiplied together to yield the power control loop parameter.

The power control loop may be configured to modulate the current driven through the energizer coil such that the power control loop parameter is maintained at approximately a predetermined target value. The predetermined target value may be based on an identification of the charger and the neurostimulator. The predetermined target value may further be based on an identification of one or more characteristics of the inductive coupling between the charger and the neurostimulator. The power control loop may be configured to duty cycle the electric current driven through the energizer coil when the change in voltage across the energizer coil is not detected. The power control loop may further comprise a pulse width modulator, wherein the pulse width modulator is configured to be duty cycled when the change in voltage across the energizer coil is not detected. The power control loop may be configured to duty cycle the electric current driven through the energizer coil when the T×H is greater than a predetermined value.

Also described herein are methods of inductively charging a microstimulator with a charger. For example a method of charging (e.g., to prevent overheating of the outside of the implant above a few degrees) may include: determining a change in voltage across an energizer coil of the charger when the energizer coil is in an unloaded state and a loaded state; and modulating a set power level of the energizer coil based on the determined changed in voltage across the energizer coil.

The power level may be modulated by setting the power level such that the power level multiplied by the change in voltage equals a predetermined value.

Any of these methods may include duty cycling the energizer coil when the change in voltage is not detected, and/or determining the charger type and the microstimulator type. The power level may be modulated by setting the power level such that the power level multiplied by the change in voltage equals a predetermined value, wherein the predetermined value is based on the charger type and the microstimulator type.

Although the implants described herein are shown and described as implantable in the neck (e.g., carotid) region of the vagus nerve, any of these implants may be inserted in other body regions, including in particular the subdiaphragmatic region (see, e.g., U.S. patent application Ser. No. 15/433,936). Furthermore, the implants described typically include batteries; in some variations any of these implants may not include a battery (e.g., “batteryless” implants that may apply stimulation in the presence of the inductive field, also described in U.S. patent application Ser. No. 15/433,936, herein incorporated by reference in its entirety).

### BRIEF DESCRIPTION OF THE DRAWINGS

FIG. 1A shows one variation of a system for modulating chronic inflammation including a leadless microstimulator (shown connected to the vagus nerve) and an external charger/controller. This apparatus may be adapted or configured to include an emergency shut-off mechanism as described herein (e.g., a static magnetic field detector) and/or it may be configured to negotiate between the charger and the implant to regulate the applied power.

FIG. 1B shows a system for modulating chronic inflammation, including a microstimulator, charger (“energizer”), and system programmer/controller (“prescription pad”).

FIG. 1C shows a system for modulating chronic inflammation, including a microstimulator, a securing device (POD) for securing the leadless stimulator to the nerve, an external charger, a system programmer/controller (“prescription pad”) and an optional surgical tester.

FIG. 1D is a block diagram schematically illustrating a microstimulator and a charger.

FIG. 2 illustrates one variation of a system including an external programmer/controller wirelessly connected to a microstimulator, which may communicate with the implant through the charger as illustrated.

FIG. 3A shows one variation of a microstimulator in a POD configured to surround a nerve of the inflammatory reflex. FIG. 3B shows an enlarged view of the microstimulator and POD. FIG. 3C shows another variation of a microstimulator; FIG. 3D shows the microstimulator of FIG. 3C within a POD. FIG. 3E shows another variation of the microstimulator.

FIG. 4 shows a schematic diagram of a microstimulator and POD around vagus nerve.

FIG. 5 is a block diagram of a static magnetic field detector for a neural microstimulator.

FIG. 6 is a flow diagram illustrating the operation of a static magnetic field detector to shut down a neural microstimulator in emergency.

FIG. 7 is a graph showing that the microcontroller temperature of the microstimulator may be proportional to the square of the microstimulator tank voltage (PWRIN); data is from two different animals having an MS implanted at the carotid.

FIG. 8 is a graph illustrating (based on thermal modeling) that surface temperatures of a microstimulator (MCU) may change as a function of power in (PWRIN); in this example, a 1.75 degrees Celsius temperature increase of the end caps occurs for a PWRIN of about 16V in some apparatuses.

FIG. 9 shows a linear relationship between the change in both microcontroller temperature (MCU) and end cap temperature as the square of PWRIN (PWRIN²).

FIGS. 10A and 10B are generic circuit descriptions of a power amplifier and an implant that receives inductive charging from the power amplifier, and a simplified circuit diagram, adapted from Ghovanloo and Atluri (“An Integrated Full-Wave CMOS Rectifier With Built-In Back Telemetry for RFID and Implantable Biomedical Applications,” Circuits and Systems I: Regular Papers, IEEE Transactions on, vol. 55, no. 10, pp. 3328-3334, November 2008). These diagrams may be helpful in describing a possible, theoretical description of the estimation of temperature in an implant from a charging device (e.g., inductive link circuit analysis), as described herein.

FIG. 11 is a graph illustrating the relationship between power in vs. margin for different coupling coefficients between the charger and the implant inductive coils.

FIG. 12A shows a first example of scaling (in a model) charger parameters to estimate temperature and/or power in (PWRIN) in an implant. FIG. 12B is a model that shows the position of the implant relative to the charger.

FIG. 13 is another example of an estimation of charger parameters illustrating estimate of power into the microcontroller may be estimated regardless of coupling coefficient, when R×U−R×L is multiplied by T×H (to give the same curve for any value of k, as T×H is a linear function of V2 where V2=0 when T×H=0).

FIG. 14 illustrates a proposed loop parameter, in which each charger size may have a unique target value for T×H(R×U−R×L), from which an estimate of PWRIN may be made.

FIG. 15A shows the relationship between PWRIN and T×H for worse-case placement of an implantable microstimulator. FIG. 15B is a graph illustrating modulating duty cycling the MS based on communication between the implant and the charger to prevent overheating of the implant.

FIG. 16 is a schematic of a controller (including a power control loop) of a microstimulator.

FIG. 17 illustrates another embodiment of a schematic of one variation of a controller for use in the microstimulator that includes a voltage-adjustable current source.

FIG. 18 schematically illustrates a system including a charger (on left) and a microstimulator implant (on right). The microcontroller may optionally include a static magnetic field detector and/or controller including adapted for regulating the power and therefore the temperature of the implant; the charger may likewise be adapted to adjust the applied energy to prevent overheating (e.g., based on the PWRIN from the microcontroller).

### DETAILED DESCRIPTION

Systems for electrically stimulating one or more nerves to treat chronic inflammation may include an implantable, wireless microstimulator such as those described herein and an external charging device (which may be referred to as a charging wand, charger, or energizer). In some variations the system also includes a controller such as a “prescription pad” that helps control and regulate the dose delivered by the system. The microstimulator may be secured in position using a securing device (which may be referred to as a “POD”) to hold the microstimulator in position around or adjacent to a nerve. These microstimulators are designed and adapted for treatment of chronic inflammation, and may be configured specifically for such use. Thus, an implantable microstimulator may be small, and adapted for the low duty-cycle stimulation to modulate inflammation. For example, the implantable microstimulator may hold a relatively small amount of power over weeks or even months and discharge it at a rate sufficient to modulate the anti-inflammatory pathway without significantly depressing heart rate or triggering any number of unwanted effects from the vagus nerve or other neural connections. Any of the nerves of the inflammatory reflex, including the vagus nerve, may be treated as described herein using the systems described.

Any of these apparatuses (systems and devices, including microstimulators and chargers) may be adapted to include one or more of an emergency shutoff, which may be based on a static magnetic field detector, and/or a thermal control or regulation based on the power in (“PWRIN”) of the microstimulator. The thermal regulator may be configured to modulate the power applied by the inductive coupling between the external charger and the microstimulator (implant), as will be described in greater detail below. In general, the microstimulator may include a power negotiation protocol. This protocol may be hardware, firmware and/or software, and may regulate charger function, including switching the charger on or off (including altering the duty cycle of the charger). The power negotiation protocol may be on the charger, the microstimulator, or both. For example, a controller of the microstimulator may execute a power negotiation protocol to negotiate at whap power the battery will charge at. The microstimulator may exchange data with and between the charger, at least in part to negotiate what power level the MS will draw power from the charger; if the MS attempts to draw too much power from the charger, it may delay or stop communication between the MS and the charger. Thus, the power negotiation protocol may throttle down the power drawn by the MS, thereby extending communication between the MR and the charger, permitting further telemetry. Initially, the charger and implant may exchange identifying information, such as charger type (capability, e.g., coil size, etc.), implant type (e.g., inductive coil size(s), etc.), coupling between the two, etc. Once communication has been initiated, the apparatus may then negotiate what level the charger will charge the implant (e.g., what applied voltage, current, power, etc.).

Any of these apparatuses may use an intermediate field communication link (e.g., when the implant is running off of a battery), including a high-gain amplifier to amplify an RF signal from the charger, using the battery rather than charging it during initial communication between the charger and implant, which may allow longer range.

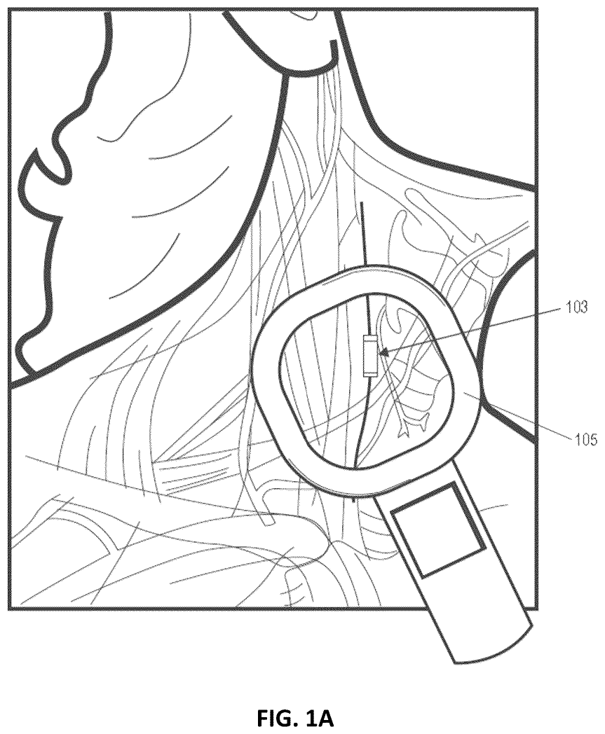

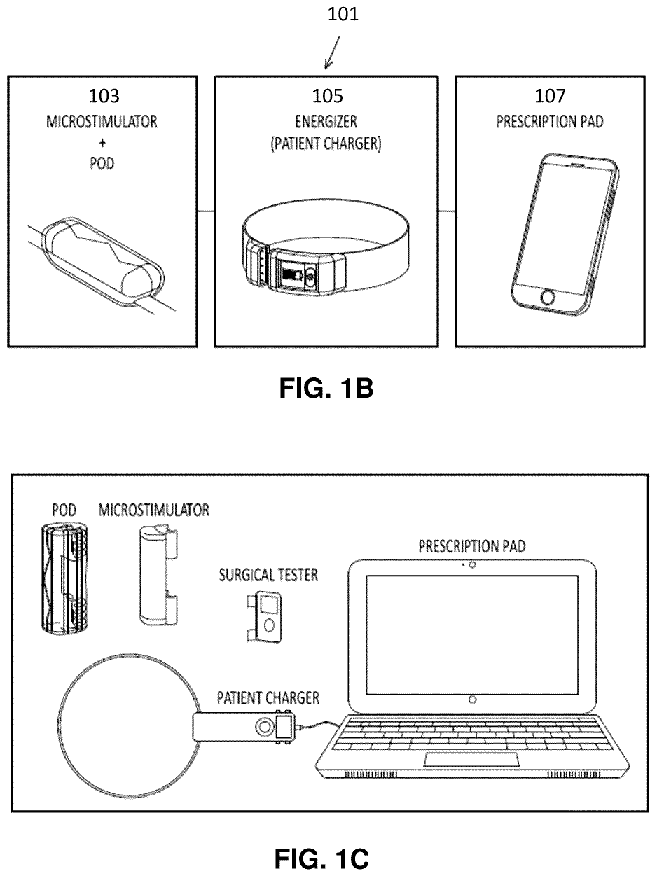

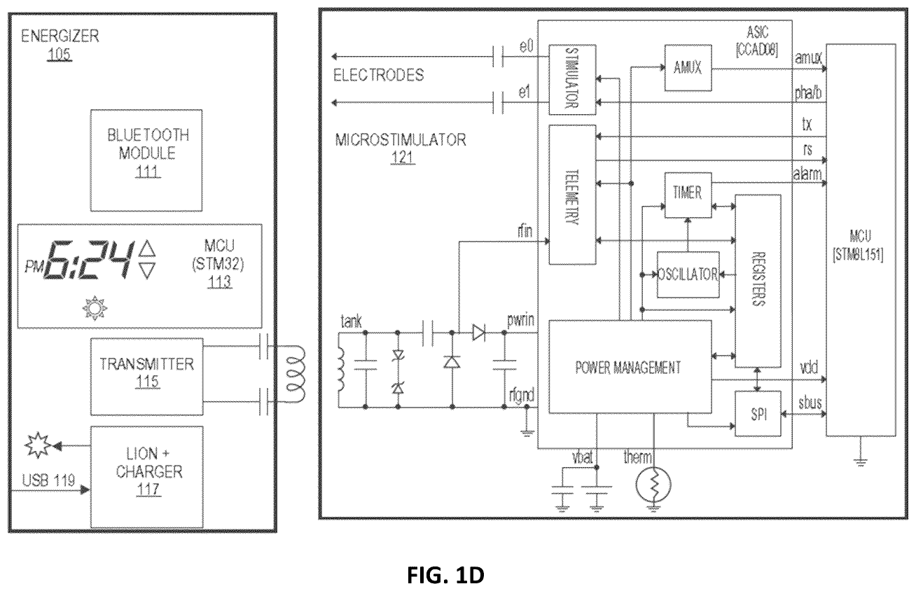

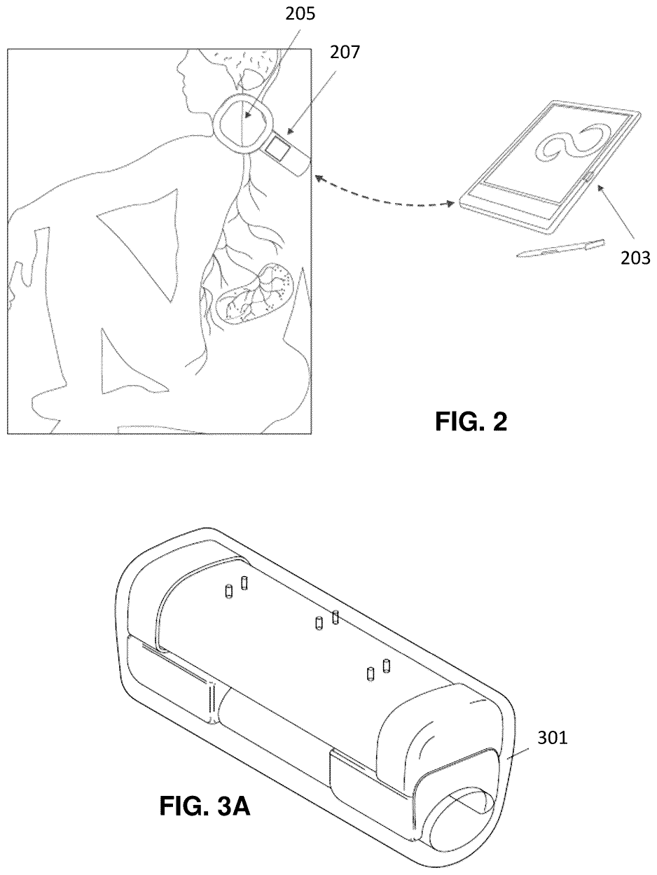

As mentioned, in general, the apparatuses described herein may include an implant (microstimulator) and a charger. For example, FIG. 1 illustrates one variation of a system for treating chronic inflammation that includes a microstimulator contained in nerve cuff (e.g., “POD”) that is mounted on cervical vagus nerve and charged a programmed by an external charger/programmer unit. This variation of a system includes a microstimulator 103 that has been implanted to contact the vagus nerve as shown. The implant may be programmed, controlled and/or charged by a charger/controller 105 device. In this variation the charger/controller is a loop with a wand region.

FIG. 1B shows another variation of a system for treating chronic inflammation that also includes an implantable microstimulator 103 (shown inserted into a POD to hold it in position relative to a nerve) and a charging device (“energizer” 105) configured as a collar to be worn around the subject's neck and charge the implant. Optionally, the system may include a separate controller (“prescription pad” 107) which may be a separate dedicated device or part of a mobile or other handheld device (e.g., an application to run on a handheld device) and may provide control information to and receive data from (e.g., telemetry) the microstimulator via the charger (or in some variations, directly).

FIG. 1C shows another variation of a system for treating chronic inflammation. The systems described herein may also be referred to as systems for the neural stimulation of the cholinergic anti-inflammatory pathway (NCAP). These systems may be configured as chronic implantable systems. In some variations, the systems are configured to treat acutely (e.g., acute may 8 hours or less), sub-acutely (expected to occur for fewer than 30 days), or chronically (expected to occur for more than 30 days).

In general, the systems described herein may be configured to apply electrical stimulation at a minimum level necessary to modulate the inflammatory reflex (e.g., modulating cytokine release) characterized by the Chronaxie and rheobase. Chronaxie typically refers to the minimum time over which an electric current double the strength of the rheobase needs to be applied in order to stimulate the neuron. Rheobase is the minimal electrical current of infinite duration that results in an action potential. As used herein, cytokines refer to a category of signaling proteins and glycoproteins that, like hormones and neurotransmitters, are used extensively in cellular communication.

The NCAP Systems described herein are typically intended for the treatment of chronic inflammation through the use of implanted neural stimulation devices (microstimulators) to affect the Neural Stimulation of the Cholinergic Anti-inflammatory Pathway (NCAP) as a potential therapeutic intervention for rheumatologic and other inflammation-mediated diseases and disorders. Neurostimulation of the Cholinergic Anti-inflammatory Pathway (NCAP) has been shown to modulate inflammation. Thus, the treatment and management of symptoms manifested from the onset of disease (e.g., inflammatory disease) is based upon the concept of modulating the Cholinergic Anti-inflammatory Pathway. The NCAP pathway normally maintains precise restraint of the circulating immune cells. As used herein, the CAP is a reflex that utilizes cholinergic nerve signals traveling via the Vagus nerve between the brain, chemoreceptors, and the reticuloendothelial system (e.g., spleen, liver). Local release of pro-inflammatory cytokines (e.g., tumor necrosis factor or TNF) from resident immune cells is inhibited by the efferent, or indirectly by afferent vagus nerve signals. NCAP causes important changes in the function and microenvironment of the spleen, liver and other reticuloendothelial organs. Leukocytes which circulate systemically become “educated” as they traverse the liver and spleen are thereby functionally down regulated by the affected environment of the reticuloendothelial system. This effect can potentially occur even in the absence of an inflammatory condition.

Under this model, remote inflammation is then dampened by down-regulated cytokine levels. Stimulation of the vagus nerve with a specific regiment of electrical pulses regulates production of pro-inflammatory cytokines. In-turn, the down regulation of these cytokines may reduce localized inflammation in joints and other organs of patients with autoimmune and inflammatory disorders.

The NCAP System includes a neurostimulator that may trigger the CAP by stimulating the cervical vagus nerve. The NCAP System issues a timed burst of current controlled pulses with sufficient amplitude to trigger the CAP at a particular interval. These two parameters, Dose Amplitude and Dose Interval, may be used by a clinician to adjust the device. For example, the clinician may set the Dose Amplitude by modifying the current level. The Dose Interval may be set by changing the duration between Doses (e.g. 12, 24, 48 hours).

In some variations, dose amplitude may be set to within the Therapy Window. The Therapy window is defined as the lower limit of current necessary to trigger the CAP, and the upper limit is the level at which the Patient feels uncomfortable. The lower limit is called the Threshold (T), and the uncomfortable level is called Upper Comfort Level (UCL).

Dose Amplitude thresholds are nonlinearly dependent upon Current (I), Pulse width (PW), Pulse Frequency (PF), and Burst Duration (BD). Amplitude is primarily set by charge (Q), that is Current (I)×Pulse width (PW). In neurostimulation applications current has the most linear relationship when determining thresholds and working within the therapy window. Therefore, the clinician may modify Dose Amplitude by modifying current. The other parameters are held to experimentally determined defaults. Pulse width is selected to be narrow enough to minimize muscle recruitment and wide enough to be well above the chronaxie of the targeted neurons. Stimulus duration and pulse frequency was determined experimentally in Preclinical work.

Dose Interval may be specific for particular diseases and the intensity of diseases experienced by a patient. Our initial research has indicated that the cervical portion of the vagus nerve may be an ideal anatomic location for delivery of stimulation. The nerve runs through the carotid sheath parallel to the internal jugular vein and carotid artery. At this location, excitation thresholds for the vagus are low, and the nerve is surgically accessible. We have not found any significant difference in biomarker modulation (e.g., modulation of cytokines) between right and left. Even though the right vagus is thought to have lower thresholds than the left in triggering cardiac dysrythmias, the thresholds necessary for NCAP are much lower than those expected to cause such dysrythmias. Therefore a device delivering NCAP can safely be applied to either the right or left vagus.

We have also found, surprisingly, that the Therapy Window is maximized on the cervical vagus through the use of a bipolar cuff electrode design. Key parameters of the cuff may be: spacing and shielding of the contacts. For example, the contact points or bands may be spaced 1-2 diameters of the vagus nerve apart, and it may be helpful to shield current from these contacts from other nearby structures susceptible to inadvertent triggering. The cuff may be further optimized by using bands which are as long and wide as possible to reduce neurostimulator power requirements.

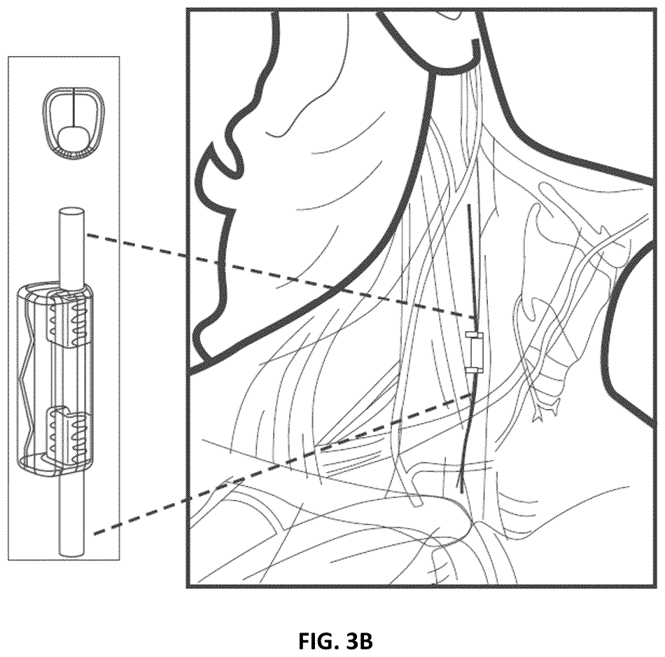

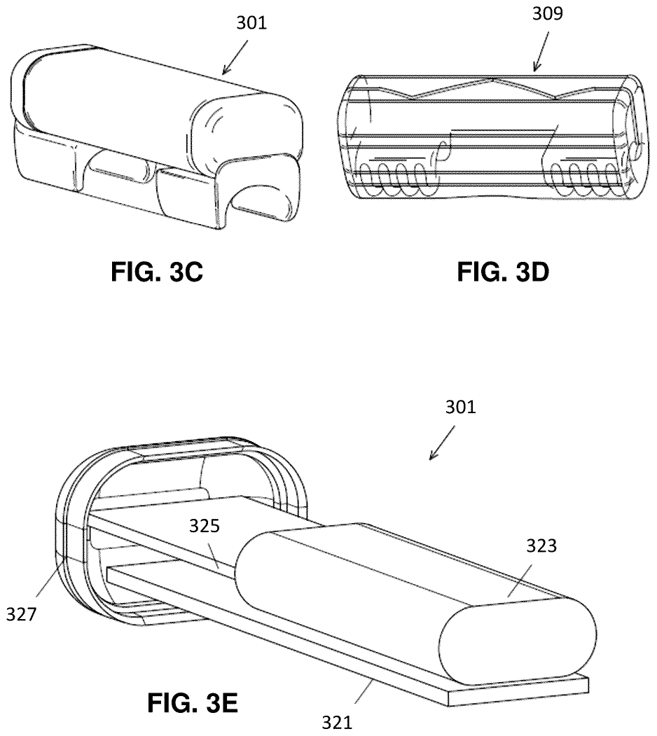

Thus, any variations of the systems described herein (e.g., the NCAP system) may be implemented with a Cuff, Lead and Implantable Pulse Generation (IPG), or a Leadless Cuff. The preferred implementation is a leadless cuff implemented by a microstimulator with integral electrode contacts in intimate contact with the nerve and contained within a Protection and Orientation Device (POD). This is illustrated in FIGS. 3A and 3B. The POD 301 may form a current shield, hold the microstimulator into place against the vagus nerve, and extend the microstimulator integral contacts with integral contacts in the POD itself. The POD is typically a polymer shell that encapsulates a microstimulator implant and that allows a nerve to run through the interior against the shell wall parallel to the length of the microstimulator implant. Within the shell of the POD, the microstimulator implant remains fixed against the Vagus nerve so the electrodes remain in contact with the nerve. The POD anchors the implant in place and prevents the implant from rotating or separating from the nerve, as well as maintaining contact between the electrodes and the nerve and preserving the orientation as necessary for efficient external charging of the microstimulator battery.

Referring back to FIG. 1C, the system may include an implantable microstimulator contained in a POD, a Patient Charger, and a prescription pad that may be used by the clinician to set dosage parameters for the patient. This system may evaluate the efficacy, safety, and usability of an NCAP technology for chronic treatment of clinical patients. The system can employ a Prescription Pad (external controller) that may include the range of treatment options.

As described in more detail in U.S. Ser. No. 12/874,171 (titled “PRESCRIPTION PAD FOR TREATMENT OF INFLAMMATORY DISORDERS”), previously incorporated by reference in its entirety, the Prescription Pad may incorporate workflows in a simplified interface and provide data collection facilities that can be transferred to an external database utilizing commercially robust and compliant methods and procedures. In use, the system may be recommended for use by a clinician after assessing a patient; the clinician may determine that treatment of chronic inflammation is warranted. The clinician may then refer the patient to an interventional doctor to implant the microstimulator. Thereafter then clinician (or another clinician) may monitor the patient and adjust the device via a wireless programmer (e.g. prescription pad). The clinician may be trained in the diagnosis and treatment procedures for autoimmune and inflammatory disorders; the interventional placement of the system may be performed by a surgeon trained in the implantation of active neurostimulation devices, with a sufficient depth of knowledge and experience regarding cervical and vagal anatomy, experienced in performing surgical dissections in and around the carotid sheath.

The system may output signals, including diagnostics, historical treatment schedules, or the like. The clinician may adjust the device during flares and/or during routine visits. Examples of implantation of the microstimulator were provided in U.S. Ser. No. 12/874,171. For example, the implant may be inserted by making an incision in the skin (e.g., ≈3 cm) along Lange's crease between the Facial Vein and the Omohyoid muscle, reflecting the Sternocleidomastoid and gaining access to the carotid sheath. The IJV may be displaced, and the vagus may be dissected from the carotid wall (≤2 cm). A sizing tool may be used to measure the vagus, and an appropriate Microstimulator and POD Kit (small, medium, large) may be selected. The POD may then be inserted under nerve with the POD opening facing the surgeon, so that the microstimulator can be inserted inside POD so that the microstimulator contacts capture the vagus. The POD may then be sutured shut. In some variations a Surgical Tester may be used to activate the microstimulator and perform system integrity and impedance checks, and shut the microstimulator off, during or after the implantation. In other variations the surgical tester may be unnecessary, as described in greater detail below.

A physician may use the Patient Charger to activate the microstimulator, perform integrity checks, and assure sufficient battery reserve exists. Electrodes may be conditioned with sub-threshold current and impedances may be measured. A Physician may charge the microstimulator. In some variations a separate charger (e.g., an “energizer”) may be used by the patient directly, separate from the controller the physician may use. Alternatively, the patient controller may include controls for operation by a physician; the system may lock out non-physicians (e.g., those not having a key, code, or other security pass) from operating or modifying the controls.

In general, a physician may establish safe dosage levels. The physician may slowly increment current level to establish a maximum limit (Upper Comfort Limit). This current level may be used to set the Dosage Level. The exact procedure may be determined during this clinical phase.

The Physician may also specify dosing parameters that specify dosage levels and dosage intervals. The device may contain several concurrent dosing programs which may be used to acclimate the patient to stimulus, gradually increase dosage until efficacy is achieved, reset tachyphylaxis, or deal with unique patient situations.

As mentioned, a patient may use the Patient Charger to replenish the microstimulator battery at necessary times (e.g., every day, every week, etc.). A clinician may also work with the patient to setup a schedule based upon the patient's stimulation needs and lifestyle. In some variations, the microstimulator battery charging is achieved by expanding the Patient Charger loop, putting the loop over the head, and closing the handle to close the loop, which may position the charger sufficiently near the implanted device. Charging may start automatically or the user (patient or physician) can push a charge button. The patient may watch the progress on the Patient Charger and may be signaled when charging is complete. The length of the charge may depend primarily upon dosage level. The more often a patient charges, the shorter the charge time may be.

The charger and/or implant may include a clock, and in some variations the patient may set the time zone on the Patient Charger to reflect his/her location. The Patient Charger may update the microstimulator time parameters while charging. This may enable the patient to adjust for travel related time zone changes or daylight savings time adjustments. Because stimulation may be perceptible (felt by the patient), it may be important that the patient receive the stimulation at the same time(s) every day.

If the patient does not charge frequently enough, the system may automatically cease treatment when about 3 months of standby battery remains. Once treatment stops the patient must visit their physician to restart treatment, to avoid damage to the implant requiring re-implantation.

In general, the microstimulator and POD can be suitable for chronic treatment with a design life of 10 years or more. The battery may support a 20 year life. Microstimulator battery charging intervals may be dependent on patient dose settings, however, as described in greater detail below, the system may be configured to conserve power and therefore minimize charging intervals and/or times, greatly enhancing patient comfort and compliance.

The microstimulator and POD may be packaged into kits. Any of the systems described herein may also include a surgical kit with the items necessary for the implantation of Microstimulator and POD. This does not prevent the surgeon, during a revision, from using the existing POD and only replacing the microstimulator. System kits may be available for small, medium, and large vagus nerves. A vagus nerve sizing kit may be available to determine which kit to use. In some variations the microstimulator and POD have a loose fit such the lumen of the device and the widest part of the nerve has a loose fit without constraining blood flow, and allowing axial flexibility and both compressive and tensile forces on the device without damaging the nerve. For example, the POD may encapsulate the microstimulator so current leakage may occur through vagus nerve access ports. All other sources of current leakage may be <25 uA when POD is sutured shut. The microstimulator may have a slot for the vagus nerve. This slot may have three sizes (approximately small, medium, large) for vagus nerves of approximately (e.g., +/−5%, 10%, 20%, 30%, 40%, 50%): 2 w×1.5 h; 3 w×2 h; 4 w×3 h (mm).

Implantable components of the microstimulator and POD and components are typically applied within the sterile barrier during the interventional procedure and may be supplied sterile. Sterilization method may be Ethylene Oxide (EO).

In some variations, the POD may be secured by 1-3 sutures and may include a marker to easily allow surgeon to match suture holes minimizing failure. The POD may be configured so that over-tightening the sutures does not cause vagal devascularization. The microstimulator and POD cross sectional area may not exceed 60 mm2 including the largest nerve model. The volume including the largest nerve model may be less than 1.5 cc.

Because rotation around the axis and movement up and down on the vagus nerve may occur during the healing period. The Patient Charger may allow accommodation of this movement.

In some variations, the microstimulator may have a bipolar stimulation current source that produce as stimulation dose with the characteristics shown in table 1, below. In some variation, the system may be configured to allow adjustment of the “Advanced Parameters” listed below; in some variations the parameters may be configured so that they are predetermined or pre-set. In some variations, the Advanced Parameters are not adjustable (or shown) to the clinician. All parameters listed in Table 1 are ±5% unless specified otherwise.

TABLE 1
Microstimulator parameters

| Property | Value | Default |
|---|---|---|
| Dosage | 0-5,000 μA in 25 μA steps | 0 |
| Amplitude (DA) | | |
| Intervals | Minute, Hour, Day, Week, | Day |
| | Month | |
| Number of | N = 60 Maximum | 1 |
| Doses per | | |
| Interval | | |

Advanced Parameters

| | | |
|---|---|---|
| Pulse width | 100-1,000 μS in 50 μS | 200 |
| Range (PW) | increments | |
| Stimulus | 1-1000 seconds | 60 |
| Duration (SD) | per dose | |
| Pulse | 1-50 Hz | 10 |
| Frequency (PF) | | |
| Stimulus | ± 3.3 or ± 5.5 ± 1 Volts | Automatically set |
| Voltage (SV) | | by software |
| Constant | ± 15% over supported range of | |
| Current Output | load impedances (200-2000Ω) | |
| Specific Dose | Set a specific time between | Driven by default |
| Time | 12:00 am-12:00 am in one | table (TBD) |
| | minute increments for each | |
| | Dose Issue | |
| Number of | 4 maximum | 1 |
| Sequential | | |
| Dosing | | |
| Programs | | |

The Dosage Interval is defined as the time between Stimulation Doses. In some variations, to support more advanced dosing scenarios, up to four ‘programs’ can run sequentially. Each program has a start date and time and will run until the next program starts. Dosing may be suspended while the Prescription Pad is in Programming Mode. Dosing may typically continue as normal while charging. Programs may be loaded into one of four available slots and can be tested before they start running. Low, Typical, and High Dose schedules may be provided. A continuous application schedule may be available by charging every day, or at some other predetermined charging interval. For example, Table 2 illustrates exemplary properties for low, typical and high dose charging intervals:

TABLE 2
low typical and high dose charging intervals

| Property | Value |
|---|---|
| Low Dose Days | 30 days max: 250 μA, 200 μS, 60 s, 24 hr, 10 |
| Charge Interval | Hz, ± 3.3 V |
| Typical Dose Charge | 30 days max: 1,000 μA, 200 μS, 120 s, 24 hr, |
| Interval | 10 Hz, ± 3.3 V |
| High Dose Charge | 3.5 days max: 5,000 μA, 500 μS, 240 s, 24 hr, |
| Interval | 20 Hz, ± 5.5 V, |

The system may also be configured to limit the leakage and maximum and minimum charge densities, to protect the patient, as shown in Table 3:

TABLE 3
safety parameters

| Property | Value | |
|---|---|---|
| Hardware DC Leakage Protection | <50 | nA |
| Maximum Charge Density | 30 | μC/cm²/phase |
| Maximum Current Density | 30 | mA/cm² |

In some variations, the system may also be configured to allow the following functions (listed in Table 4, below):

TABLE 4
Additional functions of the microstimulator and/or controller(s)

| Function | Details |
|---|---|
| Charging | Replenish Battery |
| Battery Check | Determine charge level |
| System Check | Self Diagnostics |
| Relative Temperature | Temperature difference from baseline |
| Program Management | Read/Write/Modify a dosage |
| | parameter programs |
| Program Up/Download | Transfer entire dosage parameter |
| | programs |
| Electrode Impedances | Bipolar Impedance (Complex) |
| Signal Strength | Strength of the charging signal to |
| | assist the patient in aligning the |
| | external Charge to the implanted |
| | Microstimulator. |
| Patient Parameters | Patient Information |
| Patient History | Limited programming and exception |
| | data |
| Implant Time/Zone | GMT + Time zone, 1 minute |
| | resolution, updated by Charger each |
| | charge session |
| Firmware Reload | Boot loader allows complete firmware |
| | reload |
| Emergency Stop | Disable dosing programs and |
| | complete power down system until |
| | Prescription Pad connected |

As mentioned above, in some variations, the system may record function of the microstimulator (e.g., a limited patient history). For example, the system may record: date and time that each program that is started and the associated program parameters; power down events due undercharging; hardware or software exceptions; emergency power down events; compliance events with associated impedance measurement; etc. In some variations, at least the last 50 events may be preserved in a circular buffer. Any of the systems describe herein may be approved for MRI usage at 3 Tesla (e.g., the torque will be less than a maximum threshold, the temperature rise may be less than 4° C., and the blackout area may be less than a maximum threshold volume. In some variations, the microstimulator and POD may be configured to withstand monopolar electrocautery.

The Patient Charger (including the energizer variations) typically fits over a patient's head to charge the implants in the patient's neck. As described in greater detail below, the Patient Charger may support neck circumferences ranging between 28-48 cm and head circumferences of up to 72 cm. The implant and the charger may further be configured so that they orientation of the charger and implant may allow sufficient tolerance to permit charging when worn by the user in a number of positions, without requiring substantial repositioning. The Patient Charger may provide functionality that can be accessed through a connected Prescription Pad or other external controller. For example, Table 5 below lists some function elements that may be accessed by a prescription pad in conjunction with a charger:

TABLE 5
functions that may be performed by prescription pad and charger

| | Prescription Pad | |
|---|---|---|
| | connected to | |
| Function | Charger | Charger Alone |
| Charging | Y | Y |
| Battery Check | Y | Y |
| System Check | Y | Y |
| Absolute device | Y | Used for thermal |
| Temperature | | safety purposes only |
| Program Management | Y | N |
| Program Up/Download | Y | N |
| Electrode Impedances | Y | OK Check Only |
| Signal Strength | Y | Y |
| Patient Parameters | Y | N |
| Patient History | Y | N |
| Implant | Y (time zone not | Y (synced to |
| Time/Zone/Date | changed) | Charger and |
| | | changed by patient) |
| Firmware Reload | Y | N |
| Emergency Stop | Y | Y (special |
| | | sequence) |

In general, a charger (which may be used by a patient directly) may include a recharge reminder alarm (audio and/or visual) that will remind the patient to charger on a daily, weekly, or monthly frequency. The Patient Charger may be charged through a Wall Adapter plug alone or in conjunction with a Charging Dock. The Patient Charger may clearly indicate that it is charging.

In some variations, the Patient Charger firmware will be version controlled and may be updated with Prescription Pad software in the field, or can be updated in the factory. For example, the Prescription Pad software may be controlled and may be updated in the field by the one or more web applications, a USB Dongle, a CD, etc. In some variations, the Prescription Pad may identify the microstimulator through a unique electronic ID electronically available in the microstimulator. The ID may be linked to a serial number that is embossed in the case. However, the Patient Charger may not require knowledge of this ID to charge the device.

The microstimulators described herein are configured for implantation and stimulation of the cholinergic anti-inflammatory pathway, and especially the vagus nerve. In particular the microstimulators described herein are configured for implantation in the cervical region of the vagus nerve to provide extremely low duty-cycle stimulation sufficient to modulate inflammation. These microstimulators may be adapted for this purpose by including one or more of the following characteristics, which are described in greater detail herein: the conductive capsule ends of the microstimulator may be routed to separate electrodes; the conductive capsule ends may be made from resistive titanium alloy to reduce magnetic field absorption; the electrodes may be positioned in a polymer saddle; the device includes a suspension (e.g., components may be suspended by metal clips) to safeguard the electronics from mechanical forces and shock; the device may include an H-bridge current source with capacitor isolation on both leads; the device may include a built in temperature sensor that stops energy absorption from any RF source by detuning the resonator; the device may include a built-in overvoltage sensor to stop energy absorption from any RF source by detuning resonator; the system may include DACs that are used to calibrate silicon for battery charging and protection; the system may include DACs that are used to calibrate silicon for precision timing rather than relying on crystal oscillator; the system may include a load stabilizer that maintains constant load so that inductive system can communicate efficiently; the system may include current limiters to prevent a current rush so that the microstimulator will power up smoothly from resonator power source; the system may extract a clock from carrier OR from internal clock; the device may use an ultra low power accurate RC oscillator that uses stable temperature in body, DAC calibration, and clock adjustment during charging process; the device may use a solid state LIPON battery that allows fast recharge, supports many cycles, cannot explode, and is easy to charge with constant voltage; and the device may include a resonator that uses low frequency material designed not to absorb energy by high frequency sources such as MRI and Diathermy devices.

Many of these improvements permit the device to have an extremely small footprint and power consumption, while still effectively modulating the vagus nerve.

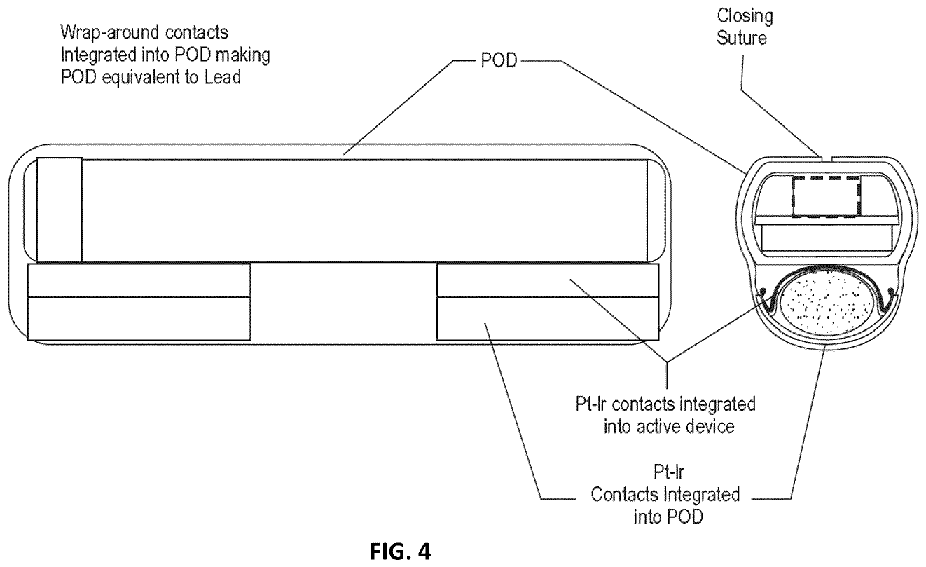

As mentioned above, some of the device variations described herein may be used with a POD to secure the implant (e.g., the leadless/wireless microstimulator implant) in position within the cervical region of the vagus nerve so that the device may be programmed and recharged by the charger/programmer (e.g., “energizer”). For example, FIG. 4 shows a schematic diagram of a POD containing a microstimulator. The cross section in FIG. 4 shows the ceramic tube containing electronic assembly that includes the hybrid, battery and coil. The rigid or semi-rigid contacts are mounted on the tube and surround the oval vagus nerve. The POD surrounds the entire device and includes a metal conductor that makes electrical contact with the microstimulator contacts and electrically surrounds the nerve.

FIG. 3A is a perspective drawing of the Pod containing the microstimulator. Sutures (not shown) are intended to be bridged across one to three sets of holes. Electrodes integrated into the pod are not shown but would extend as bands originating and ending on the two outer pairs of suture holes.

In many variations, the microstimulators described herein are tunable electrical nerve stimulators configured to deliver modulated electrical stimulus to the vagus nerve of the patient for treatment of inflammatory and autoimmune disorders. The microstimulator, in conjunction with the POD, is intended to perform as a chronic stimulating unit that generates output pulses with defined electrical characteristics to the vagus nerve of a patient. The stimulator is intended for chronic use and may be capable of executing patient specific programs with varying parameters in order to treat a wide array of diseases with differing severity levels.

In some variations, including those described above, the microstimulator consists of a ceramic body with hermetically sealed titanium-niobium ends and integral platinum-iridium electrodes attached. The microstimulator may be designed to fit within a POD 309, as shown in FIGS. 3A and 3D. As described above, the POD is a biocompatible polymer with integrated electrodes that may help the microstimulator to function as a leadless cuff electrode. In some variations, such as the variation shown in FIG. 3E, contained within the hermetic space of the microstimulator 301 is an electronic assembly that contains a rechargeable battery 321, solenoid antenna 323, hybrid circuit 325 and electrode contacts (Ti Alloy braze ring and end cap) 327 at each end to make contact with the titanium/platinum case ends.

In general, the microstimulator is designed to be implanted within deep tissue, so that it can be recharged and controlled using an external (e.g., transcutaneous) inductive link through a charger encircling the implant outside the body. One advantage to the microstimulators configured as described herein (including the extremely low duty-cycle of the device) is the low energy requirements of these devices, particularly as compared to prior art devices. For example, Table 6, below illustrates exemplary charging and use profiles for low, typical and maximally used implants. In general, the daily charging duration for low and average patients may be less than 2 minutes/day, and for Maximum patients less than 10 minutes per day.

TABLE 6
Use and charge profiles

Charge Frequency

| Patient | Full Discharge | Daily | Weekly | Monthly |
|---|---|---|---|---|
| Low | 53 days | 0.4 min | 2.6 min | 11.3 min |
| Typical | 50 days | 0.4 min | 2.8 min | 12.0 min |
| Maximum | 5 days | 3.7 min | NA | NA |

The electrode of the microstimulator may provide a nerve contact area equal to approximately ½ the surface area of the nerve for at least a length of 5 mm. Minimum electrode area=2 mm (min vagus diameter)×π×5 mm (min length)×½ circumference=15 mm2. For example, a vagus nerve diameter of 2-4 mm may be supported. In combination with the POD, less than 1 mm of total gap may be allowed around the vagus, indicating three sizes or adjustable electrodes. The bipolar Impedances of the electrodes may be less than 1000 ohms (real component).

In some variations (such as the variation illustrated above in FIG. 3E), the microstimulator may be implemented on a multi-chip hybrid substrate and consist of the following components: Microcontroller, Application Specific Integrated Circuit (ASIC), LiPON Rechargeable Battery, and various discrete components

The Patient Charger communicates with and replenishes the microstimulator deep within the body (e.g., neck) utilizing an electromagnetic field. The Patient Charger may be used by both clinicians and patients. It may include a coil that is run through a handle that separates to expand for placement over then head. Once placed over the head and closed the Patient Charger may attempt to find the Microstimulator and start charging. When charging is complete the patient may be signaled. In use, a clinician may program the microstimulator by connecting a USB cable between the Prescription Pad and the Patient Charger. The Patient Charger wirelessly connect with the implant and with outer devices (e.g., the controller such as a prescription pad). The Patient Charger may also record all charging sessions and store Microstimulator data.

The chargers described herein are configured and optimized for use with the cervical, low duty-cycle microstimulators described above, or for sub-diaphragmatic use. These charges may be worn about a subject's neck and/or waist (or torso) and may very quickly charge the implanted microstimulator, and may program and control the microstimulator as well as receive data or information from the microstimulator. The chargers described herein may use a solenoid that connects around the subject's neck with novel connection mechanism. For example, the connection mechanism may be clasp or quick connect connector that connects the loop (coil) of the charger around the subject's neck. The quick connector may be magnetic or friction fit. For example, in some variations the connector closing the loop around the subject's neck includes insertion of pins to connect one side of the loop(s) with the opposite side. For example, in some variations, the charger (e.g., energizer) coil uses a breakable coil with a magnetic latch and pogo spring pins to make contact. The coil resistance may be kept low despite the clasp/connection. For example, a multiplicity of pins may be used to keep coil resistance low and Q high.

The magnetic field strength of the charger may be modulated via a digitally compensated pwm circuit so that the power is critically tuned rather than using a resistive element. In addition, the carrier frequency may be generated using a phase accumulator to provide highly accurate frequency generation for precise tuning.

The Patient Charger may be stored in a Charging Dock to keep the battery in a charged state. A travel wall socket adapter may also be used. The Charger typically includes a battery, such as a LION rechargeable battery.

In operation, the Patient Charger may develop an axial magnetic field in alignment with the Microstimulator in the neck. The loop is sized to accommodate the largest neck and provide sufficient power to charge the battery in the adjustments to assure that sufficient charge is being transferred to Microstimulator.

Recharge time for the Microstimulator may be dependent on how much energy is drained between recharges by the patient. This may depend upon the patient settings and how often the patient charges. Patients may be able to charge as infrequently as every month. This may allow the clinician to recommend a charging schedule that is most convenient for the patient; such as when a care giver is available. The neck loop makes charging a hands free operation once the device is put around the neck.

The Energizer and Microstimulator coils may be tuned to resonate so that energy is transferred with the maximum efficiency from the Energizer to Microstimulator. The Microstimulator in turn may harvest the energy from the Energizer created magnetic field to power itself. The power harvested may be less than 15 mW. Tuning, or maximizing the mutual inductance between the two coils may be performed by using resonators that are physically adjusted to approximately 133 KHz±4 KHz. Fine tune adjustments may be made dynamically by varying the Energizer frequency with the allocated 127-135 KHz frequency band. Another method to be employed for electronic tuning that may be used induces a static flux in series inductor in the Energizer coil to electronically modify the inductance (see, e.g., U.S. Pat. No. 3,631,534).

Energy transfer may be controlled by throttling the magnetic field. The magnetic field may be created by a high efficiency Class-D amplifier. The induced coil voltage on the resonator is controlled by the collector voltage driving the amplifier. It is important to only provide sufficient power to the implant as not to saturate the Microstimulator circuits or overheat the Microstimulator, a condition which can easily be achieved. Energy transfer varies significantly with vertical position so feedback is required. Feedback may be obtained by sending a query to the Microstimulator telemeter incoming energy level. This feedback may also be obtained by measuring the difference in Energizer coil voltage between the presence and absence of the Microstimulator, as described herein. With that measure the energy being absorbed by the Microstimulator can be calculated with sufficient accuracy to control the Energizer collector voltage.

The telemetry system in this example is implemented such that two standard microprocessor UARTs communication with a RS-232 type half duplex protocol where the Energizer is the master. Two rates, e.g., 1200 and 4800 baud, may be implemented. Forward telemetry modulates the transmitter collector voltage to send data across. To keep the Microstimulator demodulator as simple as possible a DC balanced Manchester code may be employed with a simple zero crossing data slicer. The RS-232 code itself does not need to be DC balanced, but the presence of start and stop bits are sufficient to allow sufficient energy transfer during communications.

The Microstimulator resonated may be put into one of two states with a shorting switch. When the switch is open the Microstimulator is operating normally, receiving power and telemetry, and is loading the Energizer. When the Microstimulator switch is closed the coil is no longer tuned to the Energizer coil and the Microstimulator ceases to receive power, and the load that the Microstimulator normally asserts on the Energizer is removed. This switch provides several functions: it may be used to send back telemetry data to the Energizer, used by the Energizer to measure the power absorption by the Microstimulator, and/or used by the Microstimulator to turn off power absorption in case the Microstimulator becomes too hot or the internal voltage becomes too high.

The Microstimulator may respond to all packets by toggling the load switch with the UART. Data is sent in NRZ format (e.g., back telemetry). The Energizer may measure the coil voltage, removing the ≈130 KHz carrier and extracting the resulting data stream that is effectively the peak coil voltage updated at a rate of 20 KHz. The Energizer converts this analog voltage into a digital word and slices the data to produce bits that are fed to the UART. This is done in the digital domain because a sophisticated min/max peak detector can be implemented that does not require DC balanced data and can respond within 1-2 symbols.

Power is adjusted by achieving a target modulation depth on received back telemetry data. The target modulation depth is determined by calibrating the system through measurements of power transfer to the Microstimulator. It is unknown at this point when calibration will occur: once for all systems, once for each system, on power up of each system, continuously as the Energizer coil moves around.

### Static Magnetic Field Detector

Any of the implantable microstimulators described herein can include a static magnetic field detector for emergency shut off of the microstimulator. Though many safety measures have been taken, there might be unexpected situations such as circuit dysfunction or failure, sudden health condition change of the patient, unusual environment, thermistor failure, etc. A manual shut-off of the microstimulator may be needed in such emergency situations. In some variations, a magnet can be placed near the implantable microstimulator to trigger the static magnetic field detector and cause an emergency shutdown of the microstimulator.

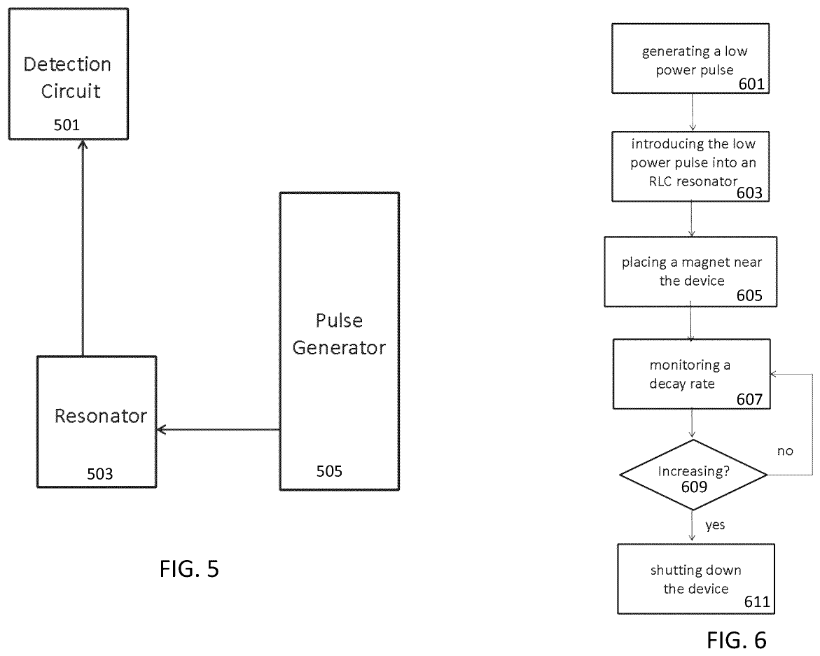

FIG. 5 is a block diagram of the static magnetic field detector for emergency shut down the microstimulator. In some variations, the static magnetic field detector can comprise a resonator built in the microstimulator. As discussed above, the implantable microstimulator can comprise an antenna to receive power from the external electric field. In some variations, the antenna can comprise a coil of wire with a ferrite core to form an inductor with a defined inductance. This inductor can be coupled with a capacitor and a resistance to form a resonant circuit (RLC circuit). The RLC circuit can have certain characteristics such a resonant frequency and a quality factor (Q). An external high quality NPO capacitor can be used to set the tank frequency. The frequency can be set to that of the radiated electric field to receive power and data from the external source. For example, the resonator can be a coil and capacitor configured to resonate at about 131 KHz+/−2%. The coil can be constructed with many turns of magnet wire with a target inductance of about 20 uH. The resonator can be configured to resonate at other frequencies and the coil can be constructed with an inductance of other values. In some other variations, it is possible that the static magnetic field detector can comprise another resonator instead of using the receiving resonator.

The static magnetic field detector can comprise a low power pulse generator. For example, a low power current pulse generator can be connected with the RLC circuit. In some variations, the power of the pulse generator can be lower than 10 microwatts. For example, the pulse generator can be an ultra-low power pulse generator based on a ring oscillator with the power between 1 nanowatt and 10 microwatts. In some other variations, the power of the pulse generator can be between 1 nanowatt and 1 microwatt. In yet some other variations, the power of the pulse generator can be lower than 1 nanowatt.

As shown, the low power pulse from the pulse generator can be introduced into the coil or the resonator RLC circuit. As known to the skilled in the art, RLC circuit can resonate at a resonance frequency or a predetermined frequency range. The oscillating current pulses decay with time due to the resistance in the circuit. When the coil is tuned, the low power pulses can take a relatively long time to decay. But when a magnetic field is introduced to the coil, the inductance of the RLC circuit is changed and the coil is detuned. Therefore, the decay rate is much quicker (or time constant is smaller). For example, a magnet can be placed near the implantable microstimulator, the ferrite of the coil can be coupled with the inductance of the static magnetic field of the magnet, the inductance of the RLC resonator can be changed to be off the resonance frequency or the predetermined frequency range. When the coil is detuned, the low power pulses take a much faster time to drop off to zero. The decay rate of the pulses can be much higher.

The static magnetic field detector can further comprise a detection circuit as shown in FIG. 5. The detection circuit 501 can be configured to monitor the decay rate of the pulses or time constant of the resonator 503. As known to the skilled in the art, the detection circuit can have many variations. For example, the detection circuit can measure a current in the resonator. For another example, the detection circuit can measure a voltage at the resonator circuit. The static magnetic field detector can be configured to shut down the microstimulator when the decay rate is increased to a predetermined value. In some variations, the static magnetic field detector can be configured to shut down the microstimulator when the decay rate is increased to 50% of the previous value. In some variations, the static magnetic field detector can be configured to shut down the microstimulator when the decay rate is increased to 50% of the previous value. In some variations, the static magnetic field detector can be configured to shut down the microstimulator when the decay rate is increased to 2 times of the previous value. The static magnetic field detector can be configured to shut down the microstimulator when the decay rate is 1.5, 2, 2.5, 3, 4, 5, 10 times of the previous value or any values therebetween. Values outside the above range are also possible.

The static magnetic field detector can be configured to shut down the microstimulator in a variety of ways. For example, the static magnetic field detector can be configured to shut down the microstimulator by using h a shorting switch. In some variations, when the switch is open, the microstimulator is operating normally; when the switch is closed, the resonator is open and the microstimulator turns off power. In some other variations, when the switch is closed, the microstimulator is operating normally; when the switch is open, the microstimulator turns off power.

In some variations, the low power pulses (e.g., from pulse generator 505) can be introduced periodically, such as every second, to detect the presence of the magnetic field. In some other variations, the low pulses can be introduced continuously to constantly monitoring the presence of the magnetic field.

Disclosed herein are also methods to shut down an implantable microstimulator by using a static magnetic field. In some variations, the method of shutting down an implantable microstimulator by the static magnetic field detector can comprise utilizing a built-in receiving coil of the microstimulator. In some other variations, the method of shutting down an implantable microstimulator by the static magnetic field detector can comprise utilizing another RLC resonator of the microstimulator.

FIG. 6 schematically illustrates the method of shutting down an implantable microstimulator by a static magnetic field detector. The method can comprise generating low power pulses 601. In some variations, the power of the pulse generator can be between 1 nanowatt and 10 microwatts. In some other variations, the power of the pulse generator can be between 1 nanowatt and 1 microwatt. In yet some other variations, the power of the pulse generator can be lower than 1 nanowatt. In some variations, the low power pulses can be introduced periodically. In some other variations, the low power pulses can be introduced continuously.

The method of shutting down an implantable microstimulator by a static magnetic field detector can further comprise introduce the pulses into the coil of the RLC resonator 603. For example, the pulses can be current pulses connected to the RLC resonator. The method can further comprise placing a magnet near the implantable microstimulator 605.

The method of shutting down an implantable microstimulator by a static magnetic field detector can further comprise monitoring the decay rate of the pulses of the resonator or the time constant of the RLC resonator 607, and determine if they are increasing or decreasing 609. When the coil is tuned, the low power pulses can take a relatively long time to decay. But when a magnetic field is introduced to the coil, the inductance of the RLC circuit is changed and the coil is detuned. Therefore, the decay rate can be much larger (or time constant can be smaller). The static magnetic field detector can be configured to shut down the microstimulator when the decay rate is at a predetermined value 611, for example, the decay rate is 1.5, 2, 2.5, 3, 4, 5, 10 times of the previous value or any values therebetween. Values outside the above range are also possible. Shutting down the device may immediately stop delivery of power to the nerve to which the implant is attached.

Any appropriate static magnetic field sensor may be used, although it may be particularly beneficial to avoid Hall effect devices. In addition to the use of a low-power pulse applied to the resonator and a detection circuit, as described above, in any of these apparatuses a Wiegand material may be used, or any other material that uses a magnetic hysteresis effect. The Wiegand effect is a nonlinear magnetic effect, and may include the use of a specially annealed and hardened wire (“Wiegand wire”), such as a low-carbon Vicalloy, a ferromagnetic alloy of cobalt, iron, and vanadium. The wire may exhibits a very large magnetic hysteresis: if a magnet is brought near the wire, the high coercivity outer shell excludes the magnetic field from the inner soft core until the magnetic threshold is reached, whereupon the entire wire both the outer shell and inner core may rapidly switch magnetization polarity (the Wiegand effect). The voltage induced by a changing magnetic field may be proportional to the rate of change of the field, thus a Wiegand-wire core can increase the output voltage of a magnetic field sensor by several orders of magnitude as compared to a similar coil with a non-Wiegand core. This higher voltage can easily be detected electronically.

### Power Control Loop

As mentioned above, in any of these apparatuses, in order to protect the patient from harm from overheating of the neurostimulator, the temperature of the outer surface of the implant can be kept within a minimum temperature range (e.g., <1.1° C., 1.3° C., 1.5° C., 1.7° C., 1.8° C., 1.9° C., 2° C., 2.1° C., 2.2° C., 2.3° C., 2.4° C., 2.5° C., etc.) of the normal surrounding body temperature. This temperature of operation is particularly critical when the implant is interacting with the charger, as described above. As will be outlined herein, the temperature of the microstimulator may be regulated within a predetermined temperature range (e.g., within about 2 degrees Celsius) by controlling the power drawn by the microstimulator from the charger.

Although the temperature may be estimated by one or more thermistor within the implant that may be used to estimate temperature, and may detune the microstimulator coil when the internal temperature reaches a threshold (internal threshold, e.g., approximately 41.5° C.), the critical temperature may be the external temperature, rather than the internal temperature. Although it may be difficult to measure the outside temperature, accurate estimation of this temperature may allow more precise and longer-lasting control of the implant, including during charging and/or telemetry, as well as enhancing safety.

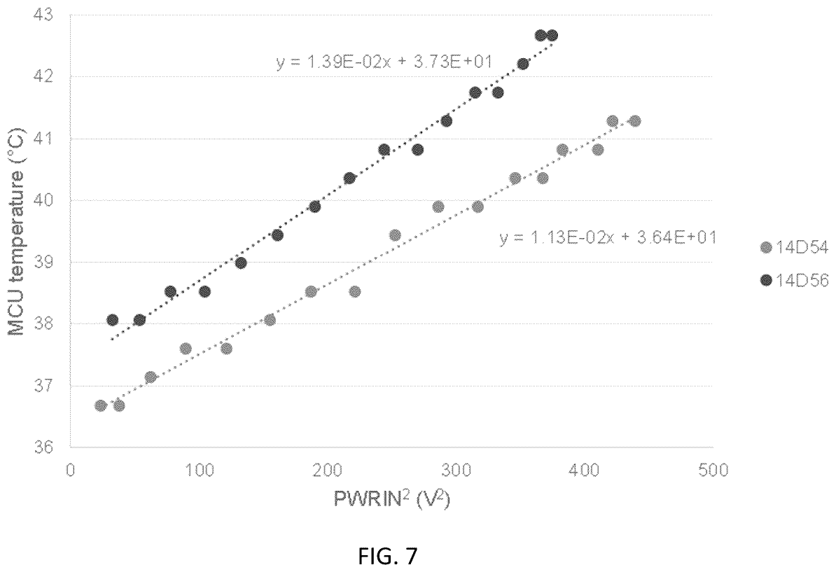

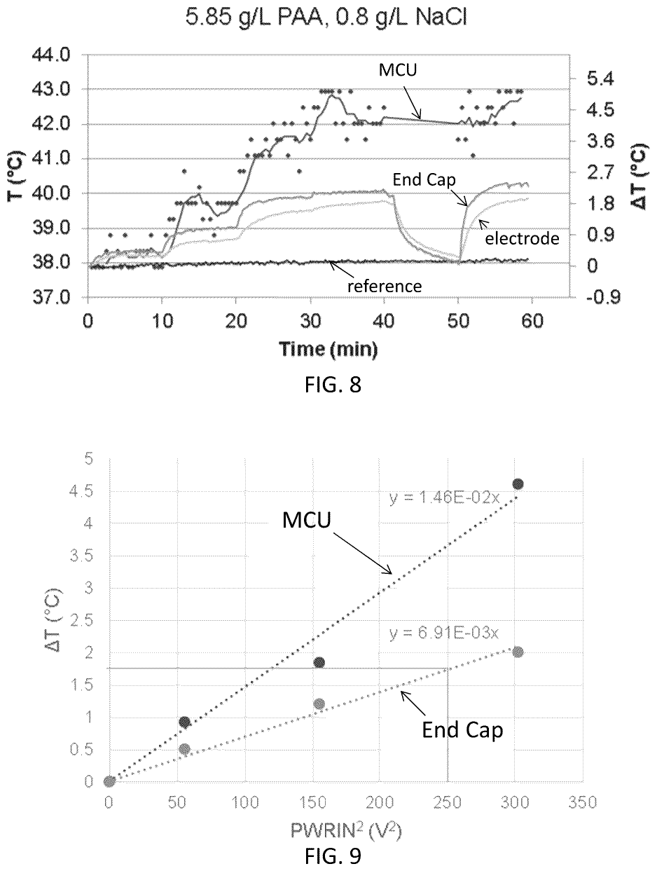

FIG. 7 shows that the microcontroller temperature of the microstimulator is proportional to the square of the microstimulator tank voltage (PWRIN). Two animal subjects were implanted with a MS and the temperature of each implant was determined, and shown to be a function of power in (PWRIN) for each. This is also shown in the graph of FIG. 8. Since the microcontroller is part of the microstimulator, the temperature of the outer surface can be estimated using a heat transfer equation, as shown in FIG. 8, which also shows the temperatures of the microcontroller and the end caps as a function of PWRIN squared. The graph in FIG. 9 shows that a 1.75 degrees Celsius temperature increase of the end caps occurs for a PWRIN of about 16V. At steady state, the rise in temperature a point i (delta Tᵢ) is related to the net heat flux, q, by the heat transfer equation:

$$q = {C}_{i} Δ {T}_{i} ,$$

Where Ci is a lumped heat loss coefficient that characterizes the heat flux path through point i into the surrounding medium. Thus, in vitro measurements show that accurate estimates of surface temperature can be determine for given power levels. If the power in (PWRIN) can be regulated (e.g., below a threshold level) then the maximum surface temperature may be regulated to <2° C. different relative to body temperature (+/−2° C.).

In operation, the PWRIN can be determined by directly querying the microstimulator (also referred to herein as the microregulator or MR). For example, the charger can query the microstimulator during charging for the PWRIN, and can adjust the power level of the charger to ensure that PWRIN remains below the desired threshold (e.g., 16V or another predetermined voltage level). However, querying the microstimulator may require a communication link between the MR and the charger and software to process and handle this information. It would be desirable if the PWRIN, the tank voltage of the MR, could be determined by values that can be measured entirely on the charger.

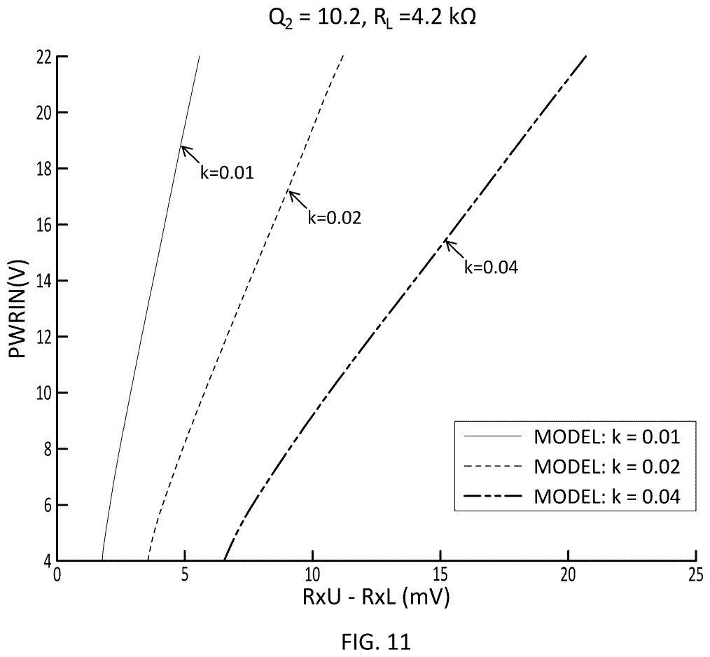

FIGS. 11-15 show an inductive circuit analysis of the charger and the MR. From this analysis, it was determined that PWRIN was correlated to the set power level of the charger (T×H) multiplied by the change in voltage across the energizer coil circuit when unloaded by the MR and when the energizer coil circuit is loaded by the MR (R×U−R×L), where R×U is the voltage across the energizer coil circuit when unloaded by the MR (can short the MR coil to create the unloaded state), and R×L is the voltage across the energizer coil circuit when loaded by the MR. This correlation holds well for any value of k, the coupling coefficient, between the charger and MR, meaning the correlation holds regardless of MR location and orientation. Thus, T×H*(R×U−R×L) can be used as part of a power control loop parameter that allows the charger to control PWRIN, and thus the increase in temperature, by monitoring only the charger parameters.

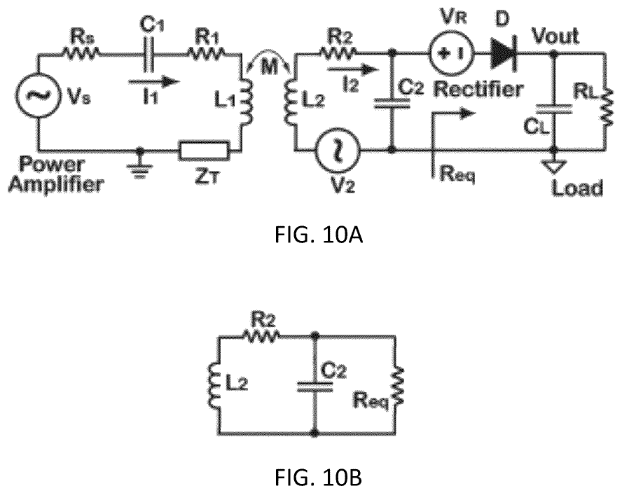

For example, FIGS. 10A-10B show an exemplary inductive charging circuit. Circuits are coupled through mutual inductance, M, where:

$$M = k \sqrt{{L}_{1} {L}_{2}} ,$$

k is the coupling coefficient. Rectifier circuit is represented as an ideal diode in this example (FIG. 10A) in series with a DC voltage source equal to the rectifier dropout voltage. It has a nonlinear impedance given by:

$${R}_{e q} = \frac{1}{2} {{R}_{L} ( \frac{{V}_{2}}{{V}_{2} - {V}_{R}} )}^{2} ,$$

Where R_{L} is the load resistance and

$${V}_{2} = {kQ}_{2} \sqrt{{L}_{1} {L}_{2}} j ω {i}_{1}$$

It may be shown that the reflected impedance, Z_{T} is given by:

$${Z}_{T} = \frac{{ω}^{2} {k}^{2} {L}_{1} {L}_{2}}{{R}_{2} + j ω {L}_{2} + \frac{{R}_{eq}}{1 + j ω {R}_{e q}}} .$$

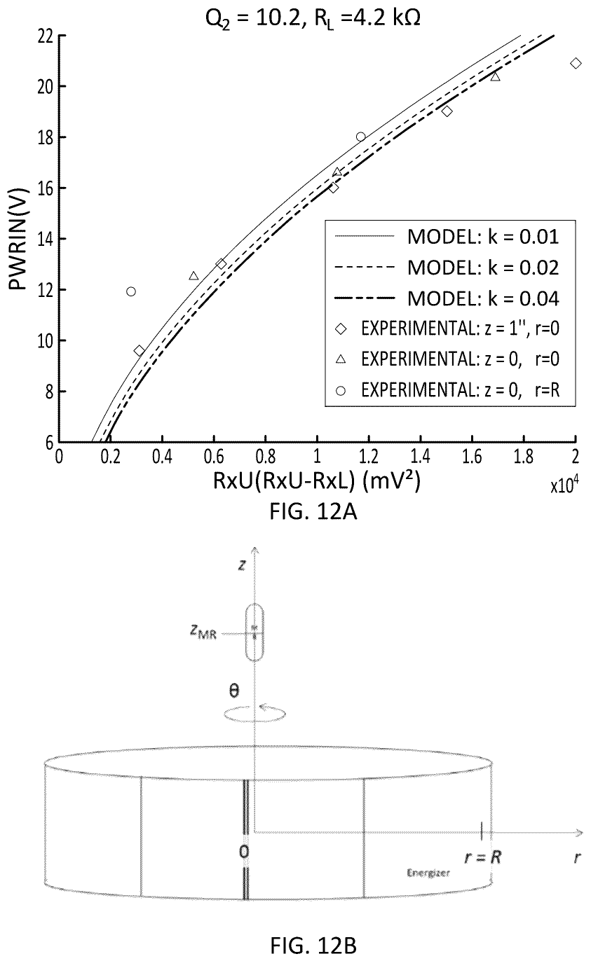

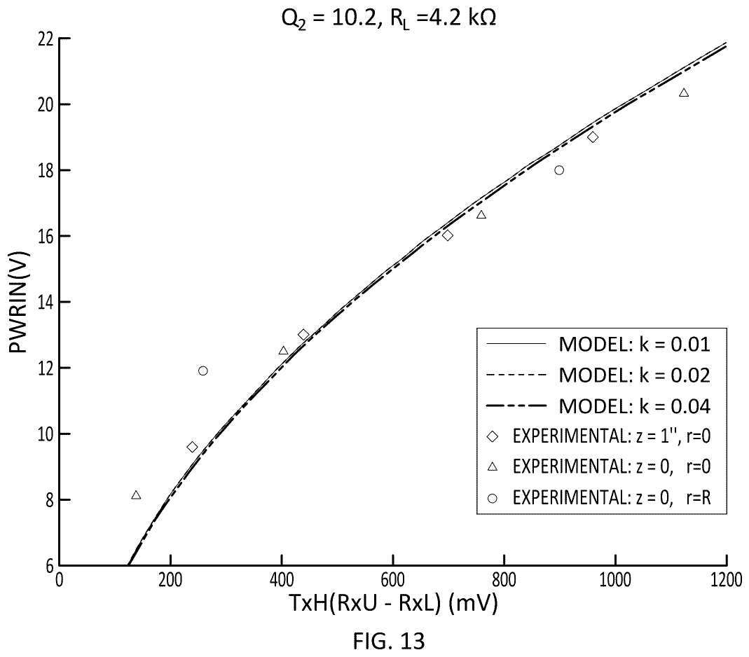

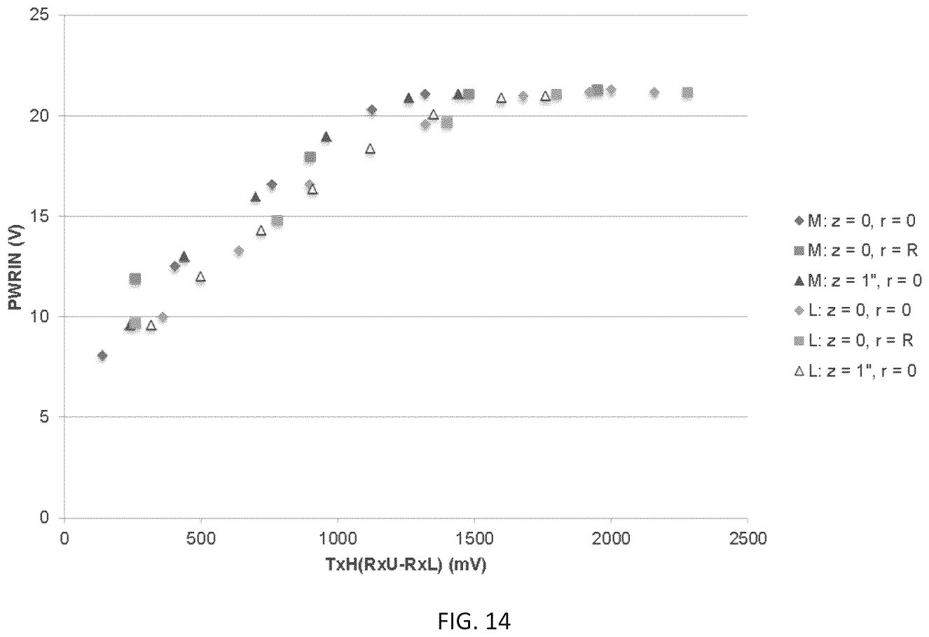

Then:

$$\vert {V}_{1} \vert = \vert {i}_{1} ( {R}_{1} + j ω {L}_{1} + {Z}_{T} ) \vert ,$$

$$or$$

$$\vert {V}_{1} \vert = \vert {V}_{1 unloaded} \vert + \vert {V}_{T} \vert ,$$

where V_{1unloaded} corresponds to R×U and V_{T} to R×U−R×l. Plotting V2=PWRN vs. margin (R×U−R×L) gives a different curve for each value of k, as shown in FIG. 11. However, it is possible to find a relationship between V₂ and V₁ that is independent of k. For example, multiplying R×U by R×U−R×L may bring the coupling curves within close alignment, as show in FIGS. 12A-12B. These curves do not match perfectly because R×U has an offset with respect to V₂ (e.g., there is a threshold V₂ for R×U=0). Alternatively, multiplying T×H by R×U−R×L may give the same curve for any value of k since T×H is a linear function of V2, where V2=0, when T×H=0, as shown in FIG. 13. The T×H value does not need to be measured, as it is being generated. Thus, using a given target value for T×H(R×U−R×L), it may be possible to determine the power in (PWRIN) within at least +/−1 V, regardless of the coupling between the implant and the charger (in the charger). Although each size of charger (e.g., coil) may have a unique target value for T×H(R×U−R×L), multiplying T×H in this manner (instead, for example, or R×U) may reduce the difference between value for different sizes, as shown in FIG. 14. Thus, the charger does not require that the communication link between the implant and the charger to query the MS for PWRIN. In any of these examples, however, the link may be active (or at least the MS may be switching and R×U−R×L may be above a detection threshold). If R×U−R×L is not detected, the implant may be configured to keep the temperature within the desired range (e.g., +/−2° C.) by duty cycling.

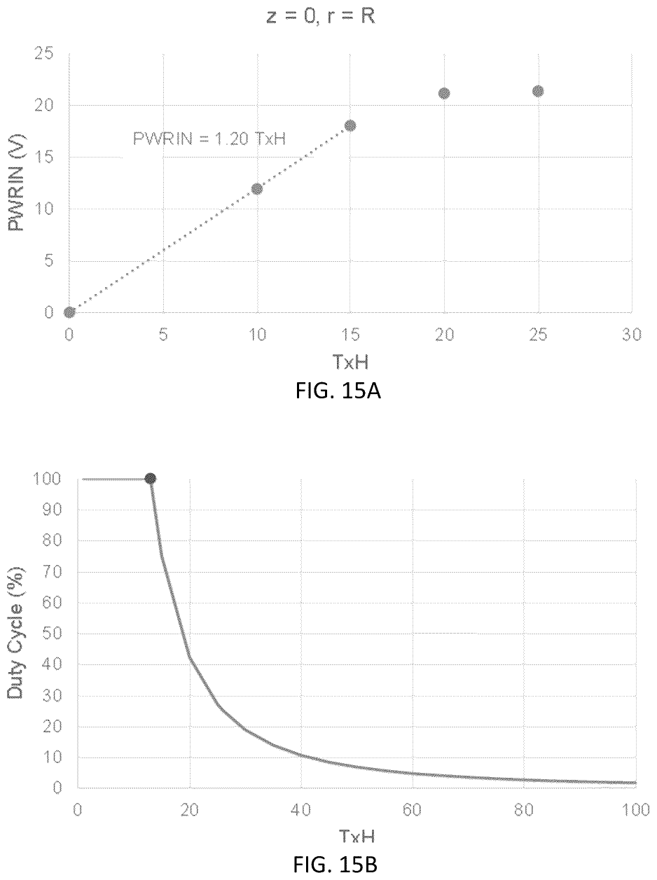

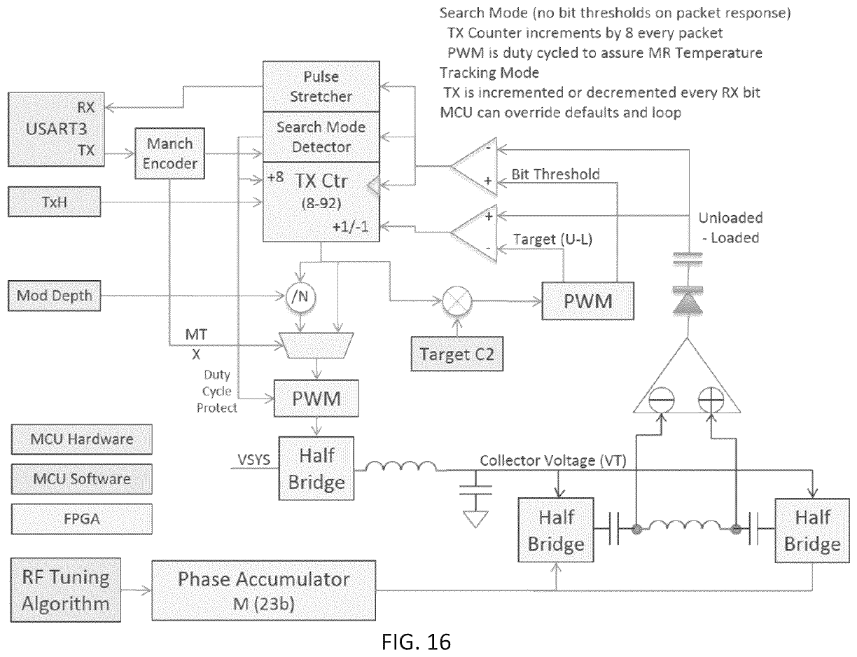

Even for worst-case placement of the microstimulator, the apparatus may determine the value of T×Hₘₐₓ that gives a target PWRIN (e.g., 16V in the examples above) to keep the temperature within a desired range. For example, for any value of T×H above T×Hₘₐₓ, the implant may use duty cycling to keep the average power constant, as shown in FIGS. 15A-15B. In this example, a duty cycle period may be picked such that, for Dₘᵢₙ (e.g., 2% duty cycle) may be chosen such that ton is sufficient to establish a communication link between the implant and the charger.

For example:

$$D = \{ \begin{matrix}100 \% , & if TxH \leq {TxH}_{max} \\ {( \frac{{TxH}_{max}}{TxH} )}^{2} \times 100 \% , & if TxH > {TxH}_{max}\end{matrix}$$

FIG. 16 illustrates schematically duty cycling in the absence of communication between the implant and the charger.

Based on this, the apparatus may determine, for a particular combination of implant and charger, a PWRIN that keeps the Tₘₐₓ of the surface of the implant within a predetermined limit (e.g., +/−2° C.). In general, MR temperature is proportional to the square of PWRIN. PWRIN may be estimated based only on measurements of energizer coil voltage, and an energizer power control loop may be based on the parameter T×H(R×U−R×L) to maintain a relatively constant value of PWRIN regardless of implant location. When the implant (microstimulator) load modulation is not detected by the charger, duty-cycling may keep the Tₘₐₓ within limit assuming the worst-case position of the microstimulator. The power control loop may be entirely hardware based, or can include firmware or software.

Thus, a target value for the power control parameter can be determined based on the type of charger, the type of implant, and the type of coupling between the two. The target values can be determined based on in vitro experiments, or can be determined in vivo during a calibration procedure. The above method of controlling the outer temperature of the MR works well except when the MR load modulation (R×U−R×L) is not detected. In this situation, the MR can be duty cycled to keep the temperature within limits, assuming the worst-case placement for MR placement, as shown in FIGS. 15A and 16. For example, a maximum value of T×H can be determined for the worst case placement that results in the target PWRIN (e.g. 16 V). When T×H of the charger is below the maximum T×H, then the charger is operated at 100% duty cycle. However, when the T×H is greater than the maximum T×H, then the charger can be operated at less than 100% duty cycle, such as according to the equation (T×Hₘₐₓ/T×H)\hat{ }2*100%.

As mentioned, a power negotiation protocol can be used by the charger and MR to determine what power level (e.g. T×H) or charge rate should be used to charge the MR. At the start of the protocol, the charging may be turned off or throttled and the communications between the charger and MR can be turned on. The initial communications can be used to exchange information regarding the type of charger (charger capabilities), the type of battery (implant capabilities), and the coupling used between the two devices, which can all be used to determine the appropriate charge rate or power level.

When the MS draws too much power, communications may collapse. Therefore, the power drawn by the MS can be throttled to restore communications. For example, in some embodiments, as the battery (e.g., a Quallion lithium-ion battery) charge is depleted, the charging current drawn from the RF tank circuit increases. This can cause antenna voltage of the implant to drop below the regulator drop out voltage in situations where insufficient RF power is available (e.g., when the implant is far from the external charger or separated from the charger by greater than a predetermined distance), which can cause communications to drop out.

In some embodiments, to mitigate this problem and prevent the antenna voltage from dropping below the regulator drop out voltage, the implant can use an adjustable current limit that restricts the amount of current drawn by the implant to perform functions outside of communication, such as charging the battery. When the implant initiates RF contact with the charger, the current limit of the implant is set to a predetermined minimum level, which presents a maximum load impedance to the RF tank circuit. If the antenna voltage of the implant is above a predetermined limit, the current limit of the implant may be increased to the next level by a predetermined amount. The process may be repeated until either the predetermined maximum current limit is reached, or the antenna voltage of the implant falls below the predetermined limit. If the antenna voltage of the implant falls below the predetermined limit, the current limit of the implant can be set at the previous level by decreasing the current limit by a predetermined amount.

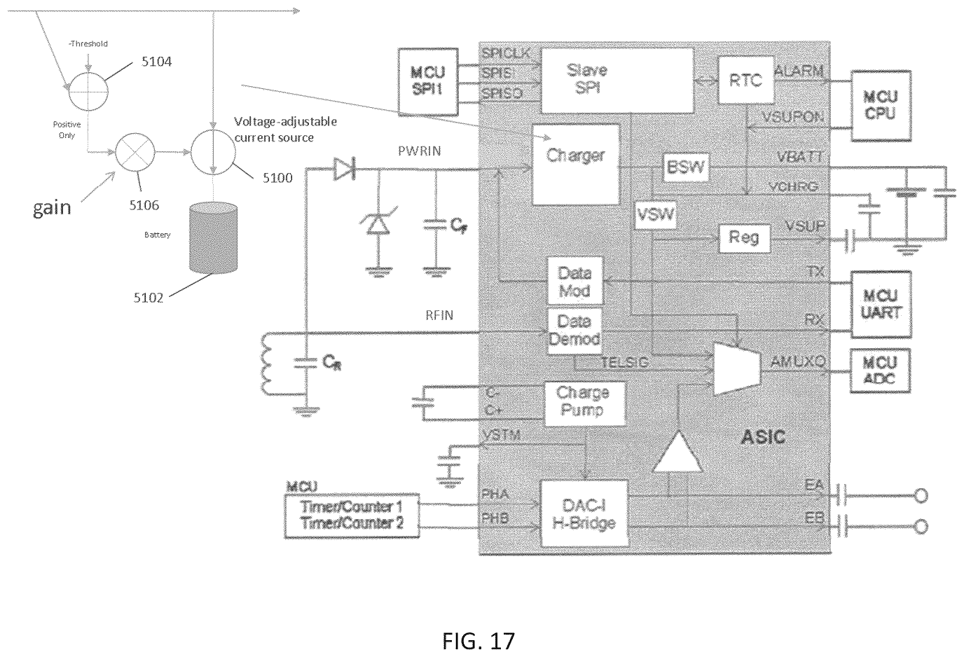

One embodiment for adjusting the current in the implant is to provide the implant with a voltage controlled current source in the charging circuit as shown in FIG. 17. The voltage controlled current source 5100 can adjust the current for charging the battery 5102 based on the antenna voltage of the implant, such that when the antenna voltage approaches and/or drops to or below a predetermined threshold, then the current is decreased or set to zero. If the antenna voltage is greater than a predetermined minimum operational threshold 5104, then the current can be increased by, for example, a multiplying factor 5106 based on the difference between the actual antenna voltage and the predetermined minimum operational threshold voltage multiplied by a gain. Below the predetermined minimum operational threshold, the current can be set to zero to preserve communication.

Prior art implants typically adjust the RF external power provided by the charger to meet the internal needs of the implant. In contrast, the implant described above is uniquely designed to modulate the power drawn by the implant, by throttling the current for example, given the RF external power provided by the charger. Such a configuration prioritizes communications over powering other functions of the implant, such as charging the battery, even though both functions may share the same circuit channel. Such a configuration also enables a “low power mode” which preserves communications even when the RF external power provided by the charger is relatively low.

Alternatively or additionally, an intermediate field communication link can be used instead of communicating through the power signal. The intermediate field communication link can be powered by the battery of the MR and use a high-gain amplifier to amplify the RF signal for transmission to the charger. This would allow the charger and MR to communicate at a longer range and enable more convenient pairing or initialization of the devices, such as before the charger is worn around the neck. For example, the patient can initiate communications while simply holding the charger in his hands, rather than having to wear the charger before initiating communications.

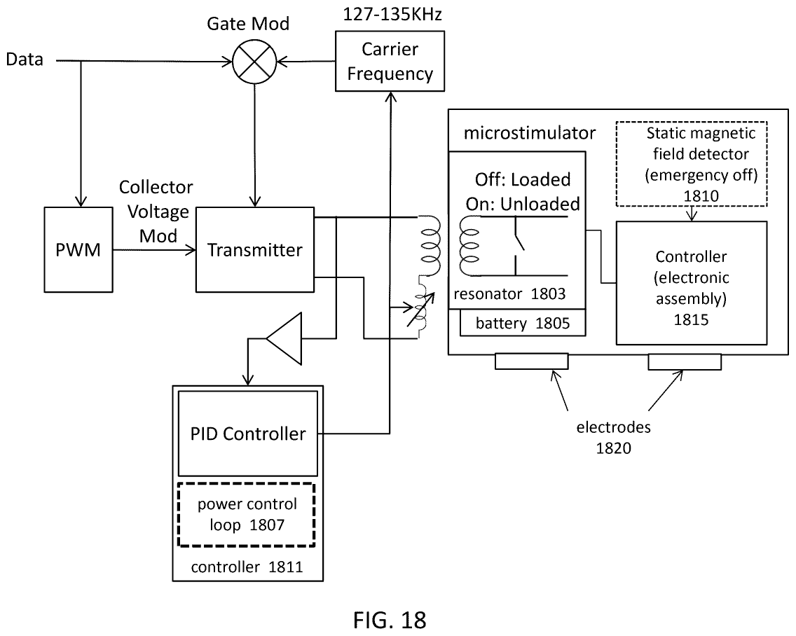

FIG. 18 schematically illustrates a charger and a microstimulator optionally incorporating both the static magnetic field detector (emergency off) 1810 and the power control loop 1807 in the charger (as part of the charger controller 1811), as discussed above. The controller may also include PID controller that uses the voltage across the two series inductors to control the carrier frequency and the inductance through a DC voltage that in turn varies the static flux in the variable inductor.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for treating chronic inflammation in a subject, comprising a leadless implantable microstimulator, the microstimulator comprising a biocompatible housing configured for implantation adjacent to neural tissue associated with an inflammatory response pathway, at least one electrode configured to deliver electrical stimulation to the neural tissue, a power receiver configured to receive energy from an external charger, a control circuit configured to generate stimulation signals for treating chronic inflammation, and a thermal management subsystem configured to regulate device temperature during operation and charging, wherein the thermal management subsystem is configured to limit a temperature increase of the microstimulator to no greater than 2° C. above surrounding tissue temperature during electrical stimulation and wireless power transfer.

2. The system of claim 1, wherein the thermal management subsystem comprises a power control loop configured to adjust received power based on a parameter correlated with implant temperature.

3. The system of claim 1, wherein the thermal management subsystem comprises a temperature sensor disposed within the housing and configured to directly measure device temperature.

4. The system of claim 1, wherein the thermal management subsystem is configured to estimate temperature based on one or more electrical characteristics of a resonant circuit within the microstimulator.

5. The system of claim 1, wherein the microstimulator is configured to temporarily reduce or suspend stimulation upon detecting a temperature exceeding a predetermined threshold.

6. The system of claim 1, wherein the microstimulator further comprises a wireless communication interface configured to transmit data indicative of at least one of device temperature or power consumption to the external charger.

7. The system of claim 1, wherein the one or more microstimulators comprise a plurality of distributed microstimulators configured to coordinate stimulation while individually maintaining the temperature increase below 2° C.

8. The system of claim 1, wherein the microstimulator is configured to stimulate a vagus nerve to modulate a cholinergic anti-inflammatory pathway.

9. The system of claim 1, wherein the housing encloses all electronic components such that no leads extend external to the housing.

10. The system of claim 1, wherein the thermal management subsystem is configured to maintain the temperature increase below 2° C. without direct temperature sensing, based solely on electrical power metrics.

11. The system of claim 1, wherein the microstimulator is configured to operate in a low-duty-cycle stimulation regime to reduce cumulative heating.

12. The system of claim 1, wherein the thermal management subsystem includes a predictive model of heat generation based on tissue coupling conditions.

13. A method of treating chronic inflammation using an implantable microstimulator, the method comprising:
implanting a leadless microstimulator proximate to a target nerve;
delivering electrical stimulation to modulate an inflammatory response;
wirelessly transferring power to the microstimulator from an external charger; and
dynamically regulating at least one of power transfer or stimulation parameters to ensure that a temperature rise of the microstimulator remains at or below 2° C. above physiological baseline.

14. The method of claim 13, further comprising estimating the temperature of the microstimulator based on power consumption characteristics.

15. The method of claim 13, wherein dynamically regulating comprises modifying at least one of stimulation amplitude, stimulation duty cycle, or charging power level.

16. A therapeutic system comprising at least one leadless implantable microstimulator and an external charger configured to inductively couple to the microstimulator, wherein the microstimulator and the charger are configured to cooperate via a feedback loop that correlates delivered power with a predicted or measured temperature of the microstimulator, and wherein the external charger is configured to adjust transmitted power to maintain a temperature rise of the microstimulator below 2° C. during charging or use.

17. The system of claim 16, wherein the external charger is configured to determine load conditions of the microstimulator and adjust transmitted power based on the load conditions.

18. The system of claim 16, wherein the external charger is configured to reduce power transmission upon detecting a threshold indicative of excessive heating.

19. The system of claim 16, wherein the external charger is configured to dynamically modulate at least one of transmission frequency, amplitude, or duty cycle to maintain the temperature rise of the microstimulator below 2° C.

20. The system of claim 16, wherein the external charger and the microstimulator are configured to perform a negotiation protocol to determine operating parameters that satisfy the temperature constraint.
